SEQUENCE LISTING (129 sequences)

SEQ ID NO: 1 - protein, 401 aa, Escherichia coli
MREAFICDGIRTPIGRYGGALSSVRADDLAAIPLRELLVRNPRLDAECIDDVILGCANQAGEDNRNVARMATLLAGLPQSVSGTTINRLCGSGLDALGFAARAIKAGDGDLLIAGGVESMSRAPFVMGKAASAFSRQAEMFDTTIGWRFVNPLMAQQFGTDSMPETAENVAELLKISREDQDSFALRSQQRTAKAQSSGILAEEIVPVVLKNKKGVVTEIQHDEHLRPETTLEQLRGLKAPFRANGVITAGNASGVNDGAAALIIASEQMAAAQGLTPRARIVAMATAGVEPRLMGLGPVPATRRVLERAGLSIHDMDVIELNEAFAAQALGVLRELGLPDDAPHVNPNGGAIALGHPLGMSGARLALAASHELHRRNGRYALCTMCIGVGQGIAMILERV

SEQ ID NO: 2 - protein, 475 aa, Escherichia coli
MMINVQTVAVIGSGTMGAGIAEVAASHGHQVLLYDISAEALTRAIDGIHARLNSRVTRGKLTAETCERTLKRLIPVTDIHALAAADLVIEAASERLEVKKALFAQLAEVCPPQTLLTTNTSSISITAIAAEIKNPERVAGLHFFNPAPVMKLVEVVSGLATAAEVVEQLCELTLSWGKQPVRCHSTPGFIVNRVARPYYSEAWRALEEQVAAPEVIDAALRDGAGFPMGPLELTDLIGQDVNFAVTCSVFNAFWQERRFLPSLVQQELVIGGRLGKKSGLGVYDWRAEREAVVGLEAVSDSFSPMKVEKKSDGVTEIDDVLLIETQGETAQALAIRLARPVVVIDKMAGKVVTIAAAAVNPDSATRKAIYYLQQQGKTVLQIADYPGMLIWRTVAMIINEALDALQKGVASEQDIDTAMRLGVNYPYGPLAWGAQLGWQRILRLLENLQHHYGEERYRPCSLLRQRALLESGYES

SEQ ID NO: 3 - protein, 255 aa, Escherichia coli
MSELIVSRQQRVLLLTLNRPAARNALNNALLMQLVNELEAAATDTSISVCVITGNARFFAAGADLNEMAEKDLAATLNDTRPQLWARLQAFNKPLIAAVNGYALGAGCELALLCDVVVAGENARFGLPEITLGIMPGAGGTQRLIRSVGKSLASKMVLSGESITAQQAQQAGLVSDVFPSDLTLEYALQLASKMARHSPLALQAAKQALRQSQEVALQAGLAQERQLFTLLAATEDRHEGISAFLQKRTPDFKGR

SEQ ID NO: 4 - DNA, 22 bp, synthetic construct
  misc_feature 1..22: note = fw primer for PaaF(L3)
  source 1..22: mol_type = other DNA
atgcaaaccatcgaaaacgaag

SEQ ID NO: 5 - DNA, 24 bp, synthetic construct
  misc_feature 1..24: note = rv primer for PaaF(L3)
  source 1..24: mol_type = other DNA
ttaacgtcctgcataggtgggttt

SEQ ID NO: 6 - DNA, 20 bp, synthetic construct
  misc_feature 1..20: note = fw primer for mmgC(l3)
  source 1..20: mol_type = other DNA
atgattcgcgaccaggaaac

SEQ ID NO: 7 - DNA, 21 bp, synthetic construct
  misc_feature 1..21: note = rv primer for mmgC(l3)
  source 1..21: mol_type = other DNA
tcaaacttggacgtcccgaat

SEQ ID NO: 8 - protein, 388 aa, unidentified
  source 1..388: note = Candida auris
MFVPTVAGKRFLTNQLRKSPQFTRMLTSHAVIFSSHGEPKDVLKTHTYEIDENNIKPNEIVLKTLGAPVNPSDINQVQGVYPSQPEKTTQLGTSEPSAVGGNEGLFEIIKIGSDVKGFQVGDWAIPTSVNFGTWRSHALCEEGKMMKVPNPEQSKQAGKKQGLTINQGATLSVNPLTAYLMLTHYVKLTPGKDWFIQNGGNSAVGKFASQIGKLLGIHSISVVRDRPEIEELKKELKEVCGATQVITEEQNNSKDFGPQVKQWVKETGGEIKLALNCVGGKSSAGVARKLAPNGLMLTYGGMSFQPVTLPTSLHIFKNVTSAGFWVTELLKSDPNLKRETVDKIVEWYTSGELLDAPSKENQWGDQDLAQVFIDAVANSKSGKQLVKF

SEQ ID NO: 9 - protein, 380 aa, unidentified
  source 1..380: note = Kluyveromyces marxianus
MSSFLSKRFISTTQRAMSQLPKAKSLIYSSHDQDVSKILKVHTYQPKGSAESSILLKTLAFPINPSDINQLEGVYPSKPEKVLDYSTEEPSAIAGNEGLFEVVSVPSGVKNLKAGDRVIPLQANFGTWSTYRTCESENDLIKIEGVDLYTAATIAVNGCTAYQMVNDYIEWDPSGNDWLVQNAGTSSVSKIVTQIAKDKGIKTLSVVRDRDNFDEVAEKLEKKYGATKVISESQNGEREFGNEVLPKILGPNAQVKLALNSVGGKSCTNIARKLSPNGLMLTYGGMSKQPVTLPTGLFIFNSIRSHGFWVTANSKRDPENKRKTVDAVVKLYRDGKIISPKEDIRTLEWDVNNLSDEGVLDLVNRGIATKGAKNMVVLKW

SEQ ID NO: 10 - protein, 373 aa, unidentified
  source 1..373: note = Pichia kudriavzevii
MSLRSVFSVSSIRRMSSITARAIVYNAHGEPKDTLKCHSYKIDLQNLQPNEVVVQTLAAPLNPSDINQIQGVYPSKPPLSIDSLPELKEPAAVTGNEGVFKVIAKGSNVGELETGDWCIPSGVNFGTWCTHRLTTDKNLLKMPKTISLNQAATIAVNPSSAYQMLTHFVKLQPGDWFIQNGANSQVGRAAIQIGHKLGYKSLNIVRNRDNLDELVKDLESIGATKVITEDESASKEFGATVKEWLEGKEIKLGLNCIGGPSASNLARKLGHDATILTYGAMSKKPLSLPTGNFIFKNLTAKGFWITGNIKRHPELRIETINAVLKFMEDGTLKEPKVNEPKVKLSTTTNESFLETVLAALEDSQKGKQLLKFE

SEQ ID NO: 11 - protein, 448 aa, unidentified
  source 1..448: note = Thermothelomyces thermophiles
MASVLSPNAAARHLLRPATRSTPPRLLPSAILHQSGNNSTRNPRISDPQSIAQRLGRRYKSGPYGYTQAKTLVFSRFGEPSDVLRLHTHSISPTLPDGAVLVRALAAPVNPADVNTIQGTYGARPAFSPLLGTPEPSAVPGNEGCFEVVAVGPRVGGGLRKGDWVIPATTGFGTFRTHALVENADRALLRVGGDKGTAGLTAKQVATVSVNPCSAYRMLKDYVDLVDLSVKSFARGDGATGGAWFLQNGANSGVGRAAIQLGRLWGLRSINVVRERATAEETEALKSELRELGATVVVTEAEFLDRSFSARLKEEWTRGDREPVMLGLNCVGGKSASAMIKALSPKGCMVTYGGMSRQSFPFPTGPQIFKRLRFEGFWLSEWAKENPAEKRNTINEILELMREGKFKESPFKEVEWNWDTEEKVLKDAIQGTLEGFKSGKGLFVFGDT

SEQ ID NO: 12 - protein, 443 aa, unidentified
  source 1..443: note = Thermothielavioides terrestris
MASALNPRHLPLRLALRPRLPLLPSTSSTTTTTTPPSCLPIPTSIRLPPRRHKSGPYGYTLAKTLVFSRFGEPRDVLSLHTHSISPALPDGAALLRALAAPVNPADVNTVQGTYGAKPAFERLLGTPEPAAVPGNEGCFEVVAIGGGAGGGGGLKKGDWVIPAQSGFGTFRTHALVEGAERKLIRVGGAKGREGLRAAQVATVSVNPCSAYRMLRDYVDLVDLSVQSFARGDGATGGAWFVQNGANSGVGRAAIQLGRLWGLRSINVVRERATPEETAALKRELAELGATVVVTESEFLDRSFADRLRDEWTRGGREPVMLGLNCVGGKSAAAMVKALSPRGCMVTYGGMSRQSFPFPTGQQIFKRLRFEGFWLSEWAKENPAAKRDTINEILELMREGKFKEAPLQEVEWNWDTEESVLKDTIQGTLEGFRPGKSIFVFKDT

SEQ ID NO: 13 - protein, 439 aa, unidentified
  source 1..439: note = Chaetomium thermophilum
MASILSPHSALRLGGISTLSRQTLLRTSRVTLTQLPKTAPQLPQRRHKSAPYGYTQSKTLVFPRFGEPIDVLSLHTHSISPTLPDTAVLLRTLAAPINPADVNTIQGTYGAKPTFSNLLGTAEPAAVPGNEGVFEVVSVGSEIAKRGVFKKGDWVIPSSSGFGTFRTHVLVEEAEQKLWRIGGEKGTEGLTPVQVATVSVNPCSAYRMLRDYVDLVGVSVRMYQEGGSDVRGGAWFLQNGANSGVGRAAIQLGRLWGLRSINVVRERATAEETEALKKELYDLGATVVVTESEFLDRSFTQRLNEEWTRGGKEPLLLALNCVGGKSAQQIVRALSPKGTMVTYGGMSRQSFPFPTGPQIFKRLRFEGFWLSEWAKENPAEKKKCVDEIIELMREGKFKEAPVQEIRWDWETEEKVLKEAVQGTLEGFRSGKGVFIFGDT

SEQ ID NO: 14 - protein, 422 aa, unidentified
  source 1..422: note = Podospora anserine
MAAPRLTSKALRLPTSKLLRPALTTATPRAAPTLTQKRHLTGPYGYHQSKALTFSSFGEPIDVLSLHTHSISPTLPSGSVLVRTLAAPVNPADVNTIQGTYGSKPPFTTLLGTAQPSAVPGNEACFEVLSVGQGVKGLEKGDWVIPAKTGFGTFRTHALVEEAEGKLMRVEREGLTPVQVATVSVNPCSAYRMLKDYVDLVGLSMRWYREGKDVSGGAWFLQNGANSGVGRAAVQFGRLWGLRSINVVRERETPEETEKLKEELTGLGANVVLTEQEFLDRSFRDRLGELTKRGKEPLLLGMNCVGGKSASAVVKALSPKGCMVTYGGMSRQSFPFPTGPQIFKRLRFEGFWLSEWGKENPEGKKKMIEDILNLMREGKFKESPVQEVEWNWETEEKTLKEAVQGTLGGFRSGKGVFVFGET

SEQ ID NO: 15 - protein, 424 aa, unidentified
  source 1..424: note = Purpureocillium lilacinum
MASSRLLGPPLRLPSSGFSRPVAALNCRAAALPLHLPRTAVRYRSGPYGYTQAKALVYSKNGEPADVLKLHTHSISPSIPSTSVLVRCLAAPINPADVNTIQGTYGSKQPLTSLIGTSEPSAVPGNEGVFEVLSAGSPSSELKKGDWVIPAASQIGTWRTHAVFGADELLKVDKEGLTPTQVSLVSVNPCTAYRILRSYGPSAGVKAALGMRPLEVGSGQWFIQNGANSGVGRAAIQFGKLWGLRSINVIRDRDTVEATDALKQELRDLGADVVVAESEFLSRGWKDQLAEITRKGREQVGLGLNCVGGKSATALARSLGEGATLVSYGGMAKQPVALPVGLLIFKDIRFVGFWLSKWNQQDVVGRRHMVADILGLMRDGKFRDAPVDEVKWDWDTDEATLRGAAQRGLEGFRQGKGVYVFGDT

SEQ ID NO: 16 - protein, 405 aa, unidentified
  source 1..405: note = Pyrenophora teres
MASKLISPSPRLAQSCLRRTRNLAPVTARFPQRRYISAYGYEQAKALTFTEYGDPSAVLSLHSHSISPPHSNYMTLRFLASPINPADINQIQGVYPSKPTFTTSLGTPNPIAVAGNEGVAEIIALGEGVKKEGFKKGDWVFMKGPGFGTWRTHASATTNDVVKLNDQMREGITAIQAGTVSINPCTAYRMLRDFTTLSEGDWFIQNGANSGVGRAAIQLGRKWGYKSINIIRSREDKNKEEAMKKELHNLGADVVITDAELQAQGIKDQAKEWTNGGRSPIRLALNCVNGKAATAMAKLLSSSAHFVTYGAMSKQPLTIPASMLIFKDIHFHGFWVSRWAEEHPEEKQKTVADVLDMTRKGEFKDMPVDEIKWEWETKGEELVAKVKDTLEGYRDGKGIFVFGKT

SEQ ID NO: 17 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 18 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSSVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 19 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 20 - protein, 1178 aa, Mycobacterium abscessus
MTETISTAAVPTTDLEEQVKRRIEQVVSNDPQLAALLPEDSVTEAVNEPDLPLVEVIRRLLEGYGDRPALGQRAFEFVTGDDGATVIALKPEYTTVSYRELWERAEAIAAAWHEQGIRDGDFVAQLGFTSTDFASLDVAGLRLGTVSVPLQTGASLQQRNAILEETRPAVFAASIEYLDAAVDSVLATPSVRLLSVFDYHAEVDSQREALEAVRARLESAGRTIVVEALAEALARGRDLPAAPLPSADPDALRLLIYTSGSTGTPKGAMYPQWLVANLWQKKWLTDDVIPSIGVNFMPMSHLAGRLTLMGTLSGGGTAYYIASSDLSTFFEDIALIRPSEVLFVPRVVEMVFQRFQAELDRSLAPGESNSEIAERIKVRIREQDFGGRVLSAGSGSAPLSPEMTEFMESLLQVPLRDGYGSTEAGGVWRDGVLQRPPVTDYKLVDVPELGYFTTDSPHPRGELRLKSETMFPGYYKRPETTADVFDDEGYYKTGDVVAELGPDHLKYLDRVKNVLKLAQGEFVAVSKLEAAYTGSPLVRQIFVYGNSERSFLLAVVVPTPEVLERYADSPDALKPLIQDSLQQVAKDAELQSYEIPRDFIVETVPFTVESGLLSDARKLLRPKLKDHYGERLEALYAELAESQNERLRQLAREAATRPVLETVTDAAAALLGASSSDLAPDVRFIDLGGDSLSALSYSELLRDIFEVDVPVGVINSVANDLAAIARHIEAQRTGAATQPTFASVHGKDATVITAGELTLDKFLDESLLKAAKDVQPATADVKTVLVTGGNGWLGRWLVLDWLERLAPNGGKVYALIRGADAEAARARLDAVYESGDPKLSAHYRQLAQQSLEVIAGDFGDQDLGLSQEVWQKLAKDVDLIVHSGALVNHVLPYSQLFGPNVAGTAEIIKLAISERLKPVTYLSTVGIADQIPVTEFEEDSDVRVMSAERQINDGYANGYGNSKWAGEVLLREAHDLAGLPVRVFRSDMILAHSDYHGQLNVTDVFTRSIQSLLLTGVAPASFYELDADGNRQRAHYDGVPGDFTAASITAIGGVNVVDGYRSFDVFNPHHDGVSMDTFVDWLIDAGYKIARIDDYDQWLARFELALKGLPEQQRQQSVLPLLKMYEKPQPAIDGSALPTAEFSRAVHEAKVGDSGEIPHVTKELILKYASDIQLLGLV

SEQ ID NO: 21 - protein, 1193 aa, Mycobacterium smegmatis
MHQLTVTGMNICEVQRLFPRMTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 22 - protein, 1178 aa, synthetic construct
  REGION 1..1178: note = MAB4714 W283RA303A
MTETISTAAVPTTDLEEQVKRRIEQVVSNDPQLAALLPEDSVTEAVNEPDLPLVEVIRRLLEGYGDRPALGQRAFEFVTGDDGATVIALKPEYTTVSYRELWERAEAIAAAWHEQGIRDGDFVAQLGFTSTDFASLDVAGLRLGTVSVPLQTGASLQQRNAILEETRPAVFAASIEYLDAAVDSVLATPSVRLLSVFDYHAEVDSQREALEAVRARLESAGRTIVVEALAEALARGRDLPAAPLPSADPDALRLLIYTSGSTGTPKGAMYPQWLVANLWQKKRLTDDVIPSIGVNFMPMSHLMGRLTLMGTLSGGGTAYYIASSDLSTFFEDIALIRPSEVLFVPRVVEMVFQRFQAELDRSLAPGESNSEIAERIKVRIREQDFGGRVLSAGSGSAPLSPEMTEFMESLLQVPLRDGYGSTEAGGVWRDGVLQRPPVTDYKLVDVPELGYFTTDSPHPRGELRLKSETMFPGYYKRPETTADVFDDEGYYKTGDVVAELGPDHLKYLDRVKNVLKLAQGEFVAVSKLEAAYTGSPLVRQIFVYGNSERSFLLAVVVPTPEVLERYADSPDALKPLIQDSLQQVAKDAELQSYEIPRDFIVETVPFTVESGLLSDARKLLRPKLKDHYGERLEALYAELAESQNERLRQLAREAATRPVLETVTDAAAALLGASSSDLAPDVRFIDLGGDSLSALSYSELLRDIFEVDVPVGVINSVANDLAAIARHIEAQRTGAATQPTFASVHGKDATVITAGELTLDKFLDESLLKAAKDVQPATADVKTVLVTGGNGWLGRWLVLDWLERLAPNGGKVYALIRGADAEAARARLDAVYESGDPKLSAHYRQLAQQSLEVIAGDFGDQDLGLSQEVWQKLAKDVDLIVHSGALVNHVLPYSQLFGPNVAGTAEIIKLAISERLKPVTYLSTVGIADQIPVTEFEEDSDVRVMSAERQINDGYANGYGNSKWAGEVLLREAHDLAGLPVRVFRSDMILAHSDYHGQLNVTDVFTRSIQSLLLTGVAPASFYELDADGNRQRAHYDGVPGDFTAASITAIGGVNVVDGYRSFDVFNPHHDGVSMDTFVDWLIDAGYKIARIDDYDQWLARFELALKGLPEQQRQQSVLPLLKMYEKPQPAIDGSALPTAEFSRAVHEAKVGDSGEIPHVTKELILKYASDIQLLGLV

SEQ ID NO: 23 - protein, 224 aa, Bacillus subtilis
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 24 - protein, 222 aa, Nocardia iowensis
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 25 - protein, 272 aa, Saccharomyces cerevisiae
MVKTTEVVSEVSKVAGVRPWAGIFVVEIQEDILADEFTFEALMRTLPLASQARILNKKSFHDRCSNLCSQLLQLFGCSIVTGLNFQELKFDKGSFGKPFLDNNRFLPFSMTIGEQYVAMFLVKCVSTDEYQDVGIDIASPCNYGGREELELFKEVFSEREFNGLLKASDPCTIFTYLWSLKESYTKFTGTGLNTDLSLIDFGAISFFPAEGASMCITLDEVPLIFHSQWFNNEIVTICMPKSISDKINTNRPKLYNISLSTLIDYFIENDGL

SEQ ID NO: 26 - protein, 209 aa, Escherichia coli
MVDMKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 27 - protein, 468 aa, Escherichia coli
MIREPPEHILNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 28 - protein, 468 aa, synthetic construct
  REGION 1..468: note = ygjG S12C6
MIREPPEHILNRLPSSASALACSAHALNLIEKRTLDHKEMKALNREVIEYYKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRRALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 29 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 30 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 31 - protein, 421 aa, Escherichia coli
MSNNEFHQRRLSATPRGVGVMCNFFAQSAENATLKDVEGNEYIDFAAGIAVLNTGHRHPDLVAAVEQQLQQFTHTAYQIVPYESYVTLAEKINALAPVSGQAKTAFFTTGAEAVENAVKIARAHTGRPGVIAFSGGFHGRTYMTMALTGKVAPYKIGFGPFPGSVYHVPYPSDLHGISTQDSLDAIERLFKSDIEAKQVAAIIFEPVQGEGGFNVAPKELVAAIRRLCDEHGIVMIADEVQSGFARTGKLFAMDHYADKPDLMTMAKSLAGGMPLSGVVGNANIMDAPAPGGLGGTYAGNPLAVAAAHAVLNIIDKESLCERANQLGQRLKNTLIDAKESVPAIAAVRGLGSMIAVEFNDPQTGEPSAAIAQKIQQRALAQGLLLLTCGAYGNVIRFLYPLTIPDAQFDAAMKILQDALSD

SEQ ID NO: 32 - protein, 464 aa, Ruegeria pomeroyi
MATITNHMPTAELQALDAAHHLHPFSANNALGEEGTRVITRARGVWLNDSEGEEILDAMAGLWCVNIGYGRDELAEVAARQMRELPYYNTFFKTTHVPAIALAQKLAELAPGDLNHVFFAGGGSEANDTNIRMVRTYWQNKGQPEKTVIISRKNAYHGSTVASSALGGMAGMHAQSGLIPDVHHINQPNWWAEGGDMDPEEFGLARARELEEAILELGENRVAAFIAEPVQGAGGVIVAPDSYWPEIQRICDKYDILLIADEVICGFGRTGNWFGTQTMGIRPHIMTIAKGLSSGYAPIGGSIVCDEVAHVIGKDEFNHGYTYSGHPVAAAVALENLRILEEENILDHVRNVAAPYLKEKWEALTDHPLVGEAKIVGMMASIALTPNKASRAKFASEPGTIGYICRERCFANNLIMRHVGDRMIISPPLVITPAEIDEMFVRIRKSLDEAQAEIEKQGLMKSAA

SEQ ID NO: 33 - DNA, 35 bp, synthetic construct
  misc_feature 1..35: note = primer for atoB
  source 1..35: mol_type = other DNA
ttgttgaataaatcggcactcggtatcgcttacct

SEQ ID NO: 34 - DNA, 35 bp, synthetic construct
  misc_feature 1..35: note = primer for atoB
  source 1..35: mol_type = other DNA
tatcgcatgcagatcggctggtggtcaacggtgcc

SEQ ID NO: 35 - DNA, 35 bp, synthetic construct
  misc_feature 1..35: note = primer for sucD
  source 1..35: mol_type = other DNA
ttgttgaataaatcggagcgcgacctggcgttgat

SEQ ID NO: 36 - DNA, 35 bp, synthetic construct
  misc_feature 1..35: note = primer for sucD
  source 1..35: mol_type = other DNA
tatcgcatgcagatcacgatatttttcagttaacc

SEQ ID NO: 37 - DNA, 35 bp, synthetic construct
  misc_feature 1..35: note = primer for ldhA
  source 1..35: mol_type = other DNA
ttgttgaataaatcgtttatcgatattgatccagg

SEQ ID NO: 38 - DNA, 35 bp, synthetic construct
  misc_feature 1..35: note = primer for ldhA
  source 1..35: mol_type = other DNA
tatcgcatgcagatctgtgtgcattacccaacggc

SEQ ID NO: 39 - DNA, 35 bp, synthetic construct
  misc_feature 1..35: note = primer for adhE
  source 1..35: mol_type = other DNA
ttgttgaataaatcgctgccgctgtctgataactg

SEQ ID NO: 40 - DNA, 35 bp, synthetic construct
  misc_feature 1..35: note = primer for adhE
  source 1..35: mol_type = other DNA
tatcgcatgcagatctcgatatacccgtactttgt

SEQ ID NO: 41 - DNA, 35 bp, synthetic construct
  misc_feature 1..35: note = primer for yqhD, dkgA
  source 1..35: mol_type = other DNA
ttgttgaataaatcgtctgtttgccgagaatacgc

SEQ ID NO: 42 - DNA, 35 bp, synthetic construct
  misc_feature 1..35: note = primer for yqhD, dkgA
  source 1..35: mol_type = other DNA
tatcgcatgcagatccgaccattttccgccaccat

SEQ ID NO: 43 - DNA, 35 bp, synthetic construct
  misc_feature 1..35: note = primer for yahK
  source 1..35: mol_type = other DNA
ttgttgaataaatcgatattcgtcctaacgaacag

SEQ ID NO: 44 - DNA, 35 bp, synthetic construct
  misc_feature 1..35: note = primer for yahK
  source 1..35: mol_type = other DNA
tatcgcatgcagatctttttgattttcaagtatgt

SEQ ID NO: 45 - DNA, 35 bp, synthetic construct
  misc_feature 1..35: note = primer for ahr
  source 1..35: mol_type = other DNA
ttgttgaataaatcgctcataacggtactgcaaac

SEQ ID NO: 46 - DNA, 35 bp, synthetic construct
  misc_feature 1..35: note = primer for ahr
  source 1..35: mol_type = other DNA
tatcgcatgcagatctcgcagcaggtaagatgatt

SEQ ID NO: 47 - DNA, 35 bp, synthetic construct
  misc_feature 1..35: note = primer for pflB
  source 1..35: mol_type = other DNA
ttgttgaataaatcgatcacctggggtcagttggc

SEQ ID NO: 48 - DNA, 35 bp, synthetic construct
  misc_feature 1..35: note = primer for pflB
  source 1..35: mol_type = other DNA
tatcgcatgcagatccgtcgttcatctgtttgaga

SEQ ID NO: 49 - DNA, 35 bp, synthetic construct
  misc_feature 1..35: note = primer for arcA
  source 1..35: mol_type = other DNA
ttgttgaataaatcgcgtagtaatgatatttcgcc

SEQ ID NO: 50 - DNA, 35 bp, synthetic construct
  misc_feature 1..35: note = primer for arcA
  source 1..35: mol_type = other DNA
tatcgcatgcagatccaccgggttagagcaaggcg

SEQ ID NO: 51 - DNA, 35 bp, synthetic construct
  misc_feature 1..35: note = primer for pdhR
  source 1..35: mol_type = other DNA
ttgttgaataaatcgcagtatttataagcaaagtg

SEQ ID NO: 52 - DNA, 35 bp, synthetic construct
  misc_feature 1..35: note = primer for pdhR
  source 1..35: mol_type = other DNA
tatcgcatgcagatcgttccagatatttcaggaat

SEQ ID NO: 53 - DNA, 35 bp, synthetic construct
  misc_feature 1..35: note = primer for gltA
  source 1..35: mol_type = other DNA
ttgttgaataaatcgaatcgcggaagcttgctcga

SEQ ID NO: 54 - DNA, 35 bp, synthetic construct
  misc_feature 1..35: note = primer for gltA
  source 1..35: mol_type = other DNA
tatcgcatgcagatcgtgtagcctgatcaatttgt

SEQ ID NO: 55 - DNA, 33 bp, synthetic construct
  misc_feature 1..33: note = primer for pHAK1
  source 1..33: mol_type = other DNA
gatctgcatgcgatatctctagaacgcgtaagc

SEQ ID NO: 56 - DNA, 28 bp, synthetic construct
  misc_feature 1..28: note = primer for pHAK1
  source 1..28: mol_type = other DNA
cgatttattcaacaaagccgccgtcccg

SEQ ID NO: 57 - DNA, 20 bp, synthetic construct
  misc_feature 1..20: note = primer for atoB
  source 1..20: mol_type = other DNA
cgatggtgattgaacggttg

SEQ ID NO: 58 - DNA, 20 bp, synthetic construct
  misc_feature 1..20: note = primer for atoB
  source 1..20: mol_type = other DNA
gcactgacgatgacacaatt

SEQ ID NO: 59 - DNA, 20 bp, synthetic construct
  misc_feature 1..20: note = primer for sucD
  source 1..20: mol_type = other DNA
gaaattcgctgctctggaag

SEQ ID NO: 60 - DNA, 23 bp, synthetic construct
  misc_feature 1..23: note = primer for sucD
  source 1..23: mol_type = other DNA
cctggcagataaccttggtgttt

SEQ ID NO: 61 - DNA, 20 bp, synthetic construct
  misc_feature 1..20: note = primer for ldhA
  source 1..20: mol_type = other DNA
tggtttaatcttgccgctcc

SEQ ID NO: 62 - DNA, 22 bp, synthetic construct
  misc_feature 1..22: note = primer for ldhA
  source 1..22: mol_type = other DNA
gtgctataaacggcgagtttca

SEQ ID NO: 63 - DNA, 26 bp, synthetic construct
  misc_feature 1..26: note = primer for adhE
  source 1..26: mol_type = other DNA
tcagtagcgctgtctggcaatataaa

SEQ ID NO: 64 - DNA, 30 bp, synthetic construct
  misc_feature 1..30: note = primer for adhE
  source 1..30: mol_type = other DNA
aatgctctcctgataatgttaaactttttt

SEQ ID NO: 65 - DNA, 25 bp, synthetic construct
  misc_feature 1..25: note = primer for yqhD
  source 1..25: mol_type = other DNA
catgcaaattctcccggtggcggta

SEQ ID NO: 66 - DNA, 24 bp, synthetic construct
  misc_feature 1..24: note = primer for yqhD
  source 1..24: mol_type = other DNA
ccaatatgagggcagagaacgatc

SEQ ID NO: 67 - DNA, 20 bp, synthetic construct
  misc_feature 1..20: note = primer for yahK
  source 1..20: mol_type = other DNA
tcgcacactaacagactgaa

SEQ ID NO: 68 - DNA, 20 bp, synthetic construct
  misc_feature 1..20: note = primer for yahK
  source 1..20: mol_type = other DNA
tgtgtttactcctgattagc

SEQ ID NO: 69 - DNA, 24 bp, synthetic construct
  misc_feature 1..24: note = primer for ahr
  source 1..24: mol_type = other DNA
cgtggtgttgaaagccgattattg

SEQ ID NO: 70 - DNA, 23 bp, synthetic construct
  misc_feature 1..23: note = primer for ahr
  source 1..23: mol_type = other DNA
cataaacttccagttctccgccc

SEQ ID NO: 71 - DNA, 32 bp, synthetic construct
  misc_feature 1..32: note = primer for pflB
  source 1..32: mol_type = other DNA
ttagatttgactgaaatcgtacagtaaaaagc

SEQ ID NO: 72 - DNA, 27 bp, synthetic construct
  misc_feature 1..27: note = primer for pflB
  source 1..27: mol_type = other DNA
gtaacacctaccttcttaagtggattt

SEQ ID NO: 73 - DNA, 27 bp, synthetic construct
  misc_feature 1..27: note = primer for arcA
  source 1..27: mol_type = other DNA
tcggctttaccaccgtcaaaaaaaacg

SEQ ID NO: 74 - DNA, 35 bp, synthetic construct
  misc_feature 1..35: note = primer for arcA
  source 1..35: mol_type = other DNA
gtttgctacctaaattgccaactaaatcgaaacag

SEQ ID NO: 75 - DNA, 35 bp, synthetic construct
  misc_feature 1..35: note = primer for pdhR
  source 1..35: mol_type = other DNA
tgatttttctggtaaaaattatccagaagatgttg

SEQ ID NO: 76 - DNA, 30 bp, synthetic construct
  misc_feature 1..30: note = primer for pdhR
  source 1..30: mol_type = other DNA
gagttcctgtcttaagccacttgccgaagt

SEQ ID NO: 77 - DNA, 28 bp, synthetic construct
  misc_feature 1..28: note = primer for gltA
  source 1..28: mol_type = other DNA
tgattgattgctaagccgtttactttcc

SEQ ID NO: 78 - DNA, 26 bp, synthetic construct
  misc_feature 1..26: note = primer for gltA
  source 1..26: mol_type = other DNA
ttaaggtctccttagcgccttattgc

SEQ ID NO: 79 - DNA, 1206 bp, Escherichia coli
  source 1..1206: mol_type = genomic DNA
atgcgtgaagcctttatttgtgacggaattcgtacgccaattggtcgctacggcggggcattatcaagtgttcgggctgatgatctggctgctatccctttgcgggaactgctggtgcgaaacccgcgtctcgatgcggagtgtatcgatgatgtgatcctcggctgtgctaatcaggcgggagaagataaccgtaacgtagcccggatggcgactttactggcggggctgccgcagagtgtttccggcacaaccattaaccgcttgtgtggttccgggctggacgcactggggtttgccgcacgggcgattaaagcgggcgatggcgatttgctgatcgccggtggcgtggagtcaatgtcacgggcaccgtttgttatgggcaaggcagccagtgcattttctcgtcaggctgagatgttcgataccactattggctggcgatttgtgaacccgctcatggctcagcaatttggaactgacagcatgccggaaacggcagagaatgtagctgaactgttaaaaatctcacgagaagatcaagatagttttgcgctacgcagtcagcaacgtacggcaaaagcgcaatcctcaggcattctggctgaggagattgttccggttgtgttgaaaaacaagaaaggtgttgtaacagaaatacaacatgatgagcatctgcgcccggaaacgacgctggaacagttacgtgggttaaaagcaccatttcgtgccaatggggtgattaccgcaggcaatgcttccggggtgaatgacggagccgctgcgttgattattgccagtgaacagatggcagcagcgcaaggactgacaccgcgggcgcgtatcgtagccatggcaaccgccggggtggaaccgcgcctgatggggcttggtccggtgcctgcaactcgccgggtgctggaacgcgcagggctgagtattcacgatatggacgtgattgaactgaacgaagcgttcgcggcccaggcgttgggtgtactacgcgaattggggctgcctgatgatgccccacatgttaaccccaacggaggcgctatcgccttaggccatccgttgggaatgagtggtgcccgcctggcactggctgccagccatgagctgcatcggcgtaacggtcgttacgcattgtgcaccatgtgcatcggtgtcggtcagggcatcgccatgattctggagcgtgtttga

SEQ ID NO: 80 - DNA, 1428 bp, Escherichia coli
  source 1..1428: mol_type = genomic DNA
atgatgataaatgtgcaaactgtggcagtgattgggagcggcaccatgggggcaggcattgctgaagttgctgccagtcatggacaccaggttttactgtatgacatttctgctgaagcgctgacccgcgcaatcgacgggatacacgcgcggctaaattcacgcgtgacgcggggaaaactgactgctgaaacctgtgaacgcacattgaaacgcctgatcccggtgaccgatattcacgcgctggcagctgcggacctggtcattgaagcggcgtctgaacgtctggaagtcaaaaaagcgctctttgcacagctggcggaagtttgcccgccacaaacgctattgaccactaacacttcgtcaatctctataaccgcgattgctgcggagataaaaaatcctgaacgtgttgcggggctgcatttttttaacccggcaccggtgatgaagttggtggaggtggtcagtgggctggcaacggcggcggaagttgttgagcagttgtgtgaactaacgttgagttggggtaagcagcctgtgcgctgtcattcgactcctggatttatcgttaaccgtgttgcgcgtccttattattccgaggcctggcgggcactggaagagcaggttgctgcaccagaagtgattgacgctgcacttcgcgatggcgctggtttcccgatggggccgctggaattaaccgatctgattggtcaggacgtcaattttgctgtcacctgttcggtgtttaacgctttctggcaggagcgtcgttttttaccttcgctggtgcaacaggaactggtgattggtggacggttgggcaagaaaagtgggctgggcgtgtacgactggcgcgcggaacgtgaggcagttgttggcctggaagcggtaagcgacagttttagcccaatgaaagtagaaaagaaaagtgacggtgtcacggaaattgacgatgttttattgattgagacacaaggcgagacggcacaggcgctggcaatacgactggcacgcccggtggtagtgatcgataaaatggcgggcaaggtggtgaccattgctgctgcagcggtgaacccggactcagcgacccgcaaggccatttattacctgcaacagcagggcaaaacagtgctgcaaattgcagattacccaggaatgctgatttggcgaacggtagcaatgatcatcaatgaagcccttgatgcgcttcaaaaaggcgtggcctctgaacaggatatcgataccgccatgcgtcttggggtgaattatccatatggcccacttgcctggggagcgcaacttggctggcagcgaatattaaggctccttgaaaatctacagcatcactatggcgaagaacgctatcgcccatgttcattgctgcgccaacgggcgcttctggagagcggttatgagtcataa

SEQ ID NO: 81 - DNA, 186 bp, synthetic construct
  misc_feature 1..186: note = promoter
  source 1..186: mol_type = other DNA
gcagctgctgcagctcgagagatctctcaagcccaaaggaagagtgaggcgagtcagtcgcgtaatgcttaggcacaggattgatttgtcgcaatgattgacacgattccgcttgacgctgcgtaaggtttgtgtataattacaggcaaccttttattcactaacaaatagctggtggaagatatc

SEQ ID NO: 82 - DNA, 16 bp, synthetic construct
  misc_feature 1..16: note = linker
  source 1..16: mol_type = other DNA
agaaggagatatacat

SEQ ID NO: 83 - DNA, 162 bp, synthetic construct
  misc_feature 1..162: note = terminator
  source 1..162: mol_type = other DNA
ggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttg

SEQ ID NO: 84 - DNA, 186 bp, synthetic construct; identical to SEQ ID NO: 81
  misc_feature 1..186: note = promoter
  source 1..186: mol_type = other DNA

SEQ ID NO: 85 - DNA, 30 bp, synthetic construct
  misc_feature 1..30: note = linker
  source 1..30: mol_type = other DNA
aagcttgcggccgcagaaggagatatacat

SEQ ID NO: 86 - DNA, 76 bp, synthetic construct
  misc_feature 1..76: note = terminator
  source 1..76: mol_type = other DNA
ctaggctgctgccaccgctgagcaataactagcataaccccttggggcctctaaacgggtcttgaggggttttttg

SEQ ID NO: 87 - DNA, 21 bp, synthetic construct
  misc_feature 1..21: note = primer for pHAK1
  source 1..21: mol_type = other DNA
ctgctgctgtcgaaaatgccg

SEQ ID NO: 88 - DNA, 23 bp, synthetic construct
  misc_feature 1..23: note = primer for pHAK1
  source 1..23: mol_type = other DNA
gaacgcggcaatttcacgatgac

SEQ ID NO: 89 - DNA, 23 bp, synthetic construct
  misc_feature 1..23: note = primer for atoB
  source 1..23: mol_type = other DNA
aagcagctaccgaaagatgcccc

SEQ ID NO: 90 - DNA, 30 bp, synthetic construct
  misc_feature 1..30: note = primer for atoB
  source 1..30: mol_type = other DNA
ggcagatgatcaaaatatctcagcgcagat

SEQ ID NO: 91 - DNA, 20 bp, synthetic construct
  misc_feature 1..20: note = primer for sucD
  source 1..20: mol_type = other DNA
gggtctggaaggtaaactgg

SEQ ID NO: 92 - DNA, 23 bp, synthetic construct
  misc_feature 1..23: note = primer for sucD
  source 1..23: mol_type = other DNA
cgtcaaagaagagcgggtcaatt

SEQ ID NO: 93 - DNA, 21 bp, synthetic construct
  misc_feature 1..21: note = primer for ldhA
  source 1..21: mol_type = other DNA
agaggttaatgcgagagagag

SEQ ID NO: 94 - DNA, 20 bp, synthetic construct
  misc_feature 1..20: note = primer for ldhA
  source 1..20: mol_type = other DNA
ctggatggtacggcgattgg

SEQ ID NO: 95 - DNA, 26 bp, synthetic construct
  misc_feature 1..26: note = primer for adhE
  source 1..26: mol_type = other DNA
tcgacatcgctatcgtcaccaccagg

SEQ ID NO: 96 - DNA, 26 bp, synthetic construct
  misc_feature 1..26: note = primer for adhE
  source 1..26: mol_type = other DNA
cgcgtataggtttgtcatcgcctgca

SEQ ID NO: 97 - DNA, 25 bp, synthetic construct
  misc_feature 1..25: note = primer for yqhDE dkgA
  source 1..25: mol_type = other DNA
tattctcaatccgtttcagcacgcg

SEQ ID NO: 98 - DNA, 24 bp, synthetic construct
  misc_feature 1..24: note = primer for yqhDE dkgA
  source 1..24: mol_type = other DNA
ggattattggccgtcttgaaggtg

SEQ ID NO: 99 - DNA, 25 bp, synthetic construct
  misc_feature 1..25: note = primer for yahK
  source 1..25: mol_type = other DNA
agctgaccatcagtcacgcctttgc

SEQ ID NO: 100 - DNA, 28 bp, synthetic construct
  misc_feature 1..28: note = primer for yahK
  source 1..28: mol_type = other DNA
cgtccagaacagagagcaataacatcac

SEQ ID NO: 101 - DNA, 20 bp, synthetic construct
  misc_feature 1..20: note = primer for ahr
  source 1..20: mol_type = other DNA
tccgctagtgtgatttcagg

SEQ ID NO: 102 - DNA, 23 bp, synthetic construct
  misc_feature 1..23: note = primer for ahr
  source 1..23: mol_type = other DNA
gaaattattatgccgccaggcgt

SEQ ID NO: 103 - DNA, 25 bp, synthetic construct
  misc_feature 1..25: note = primer for pflB
  source 1..25: mol_type = other DNA
ctgattctttgtgttgtctgcggag

SEQ ID NO: 104 - DNA, 25 bp, synthetic construct
  misc_feature 1..25: note = primer for pflB
  source 1..25: mol_type = other DNA
cagatatttagcgaactccagcgtg

SEQ ID NO: 105 - DNA, 27 bp, synthetic construct
  misc_feature 1..27: note = primer for arcA
  source 1..27: mol_type = other DNA
ctctaacagcggtaccagttctactgg

SEQ ID NO: 106 - DNA, 26 bp, synthetic construct
  misc_feature 1..26: note = primer for arcA
  source 1..26: mol_type = other DNA
gtgaaagcgcctgaagttaacggaac

SEQ ID NO: 107 - DNA, 30 bp, synthetic construct
  misc_feature 1..30: note = primer for pdhR
  source 1..30: mol_type = other DNA
aaacgtacagtacccagtagttccagccag

SEQ ID NO: 108 - DNA, 30 bp, synthetic construct
  misc_feature 1..30: note = primer for pdhR
  source 1..30: mol_type = other DNA
gaacgcgtaaacggtctgtttagaggtatc

SEQ ID NO: 109 - DNA, 25 bp, synthetic construct
  misc_feature 1..25: note = primer for gltA
  source 1..25: mol_type = other DNA
cgaatacctacgacgacgtgatacg

SEQ ID NO: 110 - DNA, 25 bp, synthetic construct
  misc_feature 1..25: note = primer for gltA
  source 1..25: mol_type = other DNA
taatccagcacgtaggtcgcattcg

SEQ ID NO: 111 - DNA, 35 bp, synthetic construct
  misc_feature 1..35: note = fw primer for paaJ(Ec) pRSFDuet
  source 1..35: mol_type = other DNA
ataaggagatataccatgcgtgaagcctttatttg

SEQ ID NO: 112 - DNA, 35 bp, synthetic construct
  misc_feature 1..35: note = rv primer for paaJ(Ec) pRSFDuet
  source 1..35: mol_type = other DNA
tgcggccgcaagctttcaaacacgctccagaatca

SEQ ID NO: 113 - DNA, 25 bp, synthetic construct
  misc_feature 1..25: note = pRSFDuet MCS1 Backbone Fw
  source 1..25: mol_type = other DNA
aagcttgcggccgcataatgcttaa

SEQ ID NO: 114 - DNA, 20 bp, synthetic construct
  misc_feature 1..20: note = pRSFDuet MCS1 Backbone Rv
  source 1..20: mol_type = other DNA
catggtatatctccttatta

SEQ ID NO: 115 - DNA, 35 bp, synthetic construct
  misc_feature 1..35: note = fw primer for paaH(Ec) pRSFDuet
  source 1..35: mol_type = other DNA
ggagatatacatatgatgataaatgtgcaaactgt

SEQ ID NO: 116 - DNA, 35 bp, synthetic construct
  misc_feature 1..35: note = rv primer for paaH(Ec) pRSFDuet
  source 1..35: mol_type = other DNA
agactcgagggtaccttatgactcataaccgctct

SEQ ID NO: 117 - DNA, 25 bp, synthetic construct
  misc_feature 1..25: note = pRSFDuet MCS2 Backbone Fw
  source 1..25: mol_type = other DNA
ggtaccctcgagtctggtaaagaaa

SEQ ID NO: 118 - DNA, 20 bp, synthetic construct
  misc_feature 1..20: note = pRSFDuet MCS2 Backbone Rv
  source 1..20: mol_type = other DNA
catatgtatatctccttctt

SEQ ID NO: 119 - DNA, 35 bp, synthetic construct
  misc_feature 1..35: note = fw primer for paaF(L3)opt pETDuet
  source 1..35: mol_type = other DNA
agaaggagatataccatgcagaccattgaaaatga

SEQ ID NO: 120 - DNA, 35 bp, synthetic construct
  misc_feature 1..35: note = rv primer for paaF(L3)opt pETDuet
  source 1..35: mol_type = other DNA
tgcggccgcaagcttttaacgaccggcataggtcg

SEQ ID NO: 121 - DNA, 25 bp, synthetic construct
  misc_feature 1..25: note = pETDuet MCS1 Backbone Fw
  source 1..25: mol_type = other DNA
aagcttgcggccgcataatgcttaa

SEQ ID NO: 122 - DNA, 25 bp, synthetic construct
  misc_feature 1..25: note = pETDuet MCS1 Backbone Rv
  source 1..25: mol_type = other DNA
catggtatatctccttcttaaagtt

SEQ ID NO: 123 - DNA, 35 bp, synthetic construct
  misc_feature 1..35: note = fw primer for ter(mtm) pETDuet
  source 1..35: mol_type = other DNA
gaaggagatatacatatggcgtcagtcttatcacc

SEQ ID NO: 124 - DNA, 35 bp, synthetic construct
  misc_feature 1..35: note = rv primer for ter(mtm) pETDuet
  source 1..35: mol_type = other DNA
tggcagcagcctaggttacgtgtcgccaaagacaa

SEQ ID NO: 125 - DNA, 1347 bp, unidentified
  source 1..1347: mol_type = genomic DNA; note = Thermothelomyces thermophilus
atggcgtcagtcttatcaccaaacgcagcagcacgtcatctgctgcgtcctgctacccggtctacaccgcctcgtttactgccgtccgcaatcttacaccaaagcggcaataactccacgcgcaatccgcgcattagcgatccgcagtcaattgctcaacggttaggtcgtcgctacaaatcgggcccgtacggctatacgcaggccaaaacgctggtgttttcgcgtttcggagaaccctccgatgttctgcgcctgcatacgcatagcatcagtccgacactgccggatggtgcggttctggtgcgggcactggcagctccagtaaacccggcggatgtcaacactatccaaggcacctatggtgcgcgtccagctttcagccccttactcggcactcccgaacctagtgcggttccaggcaacgaaggttgctttgaagttgtcgcagttggtccacgtgtcggcggcggtctgcgtaaaggggattgggtgattccggccaccaccggctttggcacctttcgcacacacgcgttggtggagaatgccgatcgcgctctgcttcgcgtaggaggtgacaaaggtaccgcgggtctgaccgccaaacaggtggccacggtaagcgtcaatccgtgttcggcctatcgtatgctcaaggactacgtcgatctggtggacttgagtgtgaagtcctttgctcgcggtgatggggccactggcggggcgtggtttctccagaatggcgcgaatagcggagttggtcgcgcggcgattcagctgggccgcctctggggtctgcgctcgattaacgtggttcgtgaacgcgctaccgccgaagaaaccgaagcattgaaaagcgaactgcgtgagcttggtgcgacggttgttgtgaccgaagcggaattccttgatcgctctttctcggcccgcttgaaagaagagtggacacgcggcgatcgcgaaccggtgatgctgggtttgaactgtgtaggcgggaaaagcgcgagcgccatgatcaaggccctgtctccgaaaggttgcatggtgacttatggcgggatgagtcgtcagtcattcccctttccgactggtcctcagatctttaaacgcctgcgttttgagggattctggctgagtgaatgggccaaagagaatccggcggagaaacgcaacaccattaatgagattctggagctgatgcgggaaggcaaattcaaagagtctccgttcaaagaagtggaatggaactgggacacggaagaaaaggtactgaaagacgcaattcaaggcacccttgaagggttcaagtccgggaaaggattatttgtctttggcgacacgtaa

SEQ ID NO: 126 - DNA, 25 bp, synthetic construct
  misc_feature 1..25: note = pETDuet MCS2 Backbone Fw
  source 1..25: mol_type = other DNA
cctaggctgctgccaccgctgagca

SEQ ID NO: 127 - DNA, 25 bp, synthetic construct
  misc_feature 1..25: note = pETDuet MCS2 Backbone Rv
  source 1..25: mol_type = other DNA
catatgtatatctccttcttatact

SEQ ID NO: 128 - DNA, 765 bp, Escherichia coli
  source 1..765: mol_type = genomic DNA
atggcctacagcaaaatccgccaaccaaaactctccgatgtgattgagcagcaactggagtttttgatcctcgaaggcactctccgcccgggcgaaaaactcccaccggaacgcgaactggcaaaacagtttgacgtctcccgtccctccttgcgtgaggcgattcaacgtctcgaagcgaagggcttgttgcttcgtcgccagggtggcggcacttttgtccagagcagcctatggcaaagcttcagcgatccgctggtggagctgctctccgaccatcctgagtcacagtatgacttgctcgaaacacgacacgccctggaaggtatcgccgcttattacgccgcgctgcgtagtaccgatgaagacaaggaacgcatccgtgaactccaccacgccatagagctggcgcagcagtctggcgatctggacgcggaatcaaacgccgtactccagtatcagattgccgtcaccgaagcggcccacaatgtggttctgcttcatctgctaaggtgtatggagccgatgttggcccagaatgtccgccagaacttcgaattgctctattcgcgtcgcgagatgctgccgctggtgagtagtcaccgcacccgcatatttgaagcgattatggccggtaagccggaagaagcgcgcgaagcatcgcatcgccatctggcctttatcgaagaaattttgctcgacagaagtcgtgaagagagccgccgtgagcgttctctgcgtcgtctggagcaacgaaagaattag

SEQ ID NO: 129 - protein, 254 aa, Escherichia coli [sequence omitted]

(19) United States
(12) Patent Application Publication
(10) Pub. No.: US 2023/0227865 A1
(43) Pub. Date: Jul. 20, 2023

(54) Modified Microorganisms and Production Method of Compounds

(71) Applicant: Asahi Kasei Kabushiki Kaisha, Tokyo (JP)

(72) Inventors: Yutaro Yamada, Tokyo (JP); Ryo Miyatake, Tokyo (JP); Koji Isaka, Tokyo (JP)

(73) Assignee: Asahi Kasei Kabushiki Kaisha, Tokyo (JP)

(21) Appl. No.: 18/152,907

(22) Filed: Jan. 11, 2023

(30) Foreign Application Priority Data
Jan. 19, 2022 (JP) 2022-006726

(51) Int. Cl. C12P 7/44; C12N 9/02

(52) U.S. Cl. CPC C12P 7/44; C12N 9/0008; C12Y 102/04001

(57) ABSTRACT

A modified microorganism containing a genetic modification that suppresses a transcription factor that controls expression of pyruvate dehydrogenase, in which the microorganism has and a production pathway of a C6 compound, and in which the C6 compound is at least one compound selected from the group consisting of adipic acid, hexamethylenediamine, 1,6-hexanediol, 6-aminohexanoic acid, 6-amino-1-hexanol, 6-hydroxyhexanoic acid, 3-oxoadipic acid, 3-hydroxyadipic acid, and 2,3-dehydroadipic acid.

### BACKGROUND OF THE INVENTION

### 1. Technical Field

The present invention relates to a modified microorganism and a method for producing a compound.

### 2. Description of the Related Art

A C6 compound such as adipic acid, hexamethylenediamine, 1,6-hexanediol, 6-aminohexanoic acid, 6-amino-1-hexanol and 6-hydroxyhexanoic acid is used, for example, as a raw material for a polyamide, a polyurethane or the like, and is regarded as an important compound in the chemical industry. Recently, since fossil fuels are in danger of depletion and are considered to be a cause of global warming. Therefore, in a chemical production process, it is desired to shift from raw materials derived from fossil fuels to renewable raw materials, such as raw materials derived from biomass, and a method for producing a compound by fermentation production of microorganisms whose metabolism is modified by gene recombination has been proposed.

On the other hand, in the production process using microorganisms, the insufficiency of productivity can be a problem. In addition, in the production process using microorganisms, one of the problems is that by-products such as a compound which is originally produced by microorganisms and is different from a target compound and a compound produced by a side reaction from a metabolic intermediate can be produced. The production of the by-products contributes to a decrease in yield of the target compound, and further contributes to an increase in load and cost in a separation and purification process. Therefore, various studies have been conducted to improve the productivity of the target compound and reduce the production of the by-products. As such a technique, a genetic modification of a host microorganism as described below has been proposed.

For example, an adipic acid-producing microorganism and an adipic acid-production example in which Escherichia coli in which ldhA, poxB, pta, adhE, and sucD genes are disrupted is used as a host and an adipic acid biosynthetic gene is introduced into the host have been proposed (Cheong, Seokjung, James M. Clomburg, and Ramon Gonzalez. “Energy-and carbon-efficient synthesis of functionalized small molecules in bacteria using non-decarboxylative Claisen condensation reactions” “Nature biotechnology” 34.5 (2016): 556-561).

In addition, for example, a gene deletion set that can increase production of a compound such as 6-aminocaproic acid, hexamethylenediamine, and adipic acid estimated by an in silico method is disclosed (JP 5773990 B2).

Furthermore, for example, in a genetic recombinant microorganism containing a pathway for producing hexamethylenediamine, 6-aminohexanoic acid, adipic acid, caprolactam, caprolactone, levulinic acid, and 1,6-hexanediol, a genetic modification capable of suppressing by-products has been proposed (WO 2016/209883 A).

However, the modification examples of the host microorganism according to the related art described above are limited to a genetic modification of an enzyme that promotes a chemical reaction directly involved in production of a target compound or a by-product. The microorganism containing the C6 compound production pathway according to the related art described above does not necessarily provide sufficient productivity for contributing to industrial use, and thus it has been required to improve a production ability of the microorganism.

### SUMMARY OF THE INVENTION

An object of the present invention is to provide a novel modified microorganism having a C6 compound production ability and a novel method for producing a C6 compound.

As a result of conducting studies, the present inventors have focused on a transcription factor that controls gene expression in addition to a method for directly modifying an enzyme gene involved in a metabolic pathway reaction, and have found that a C6 compound production ability can be improved by suppressing a specific transcription factor in a microorganism containing a C6 compound production pathway. Furthermore, it was found that production of valine accumulated as a by-product could be reduced by suppressing the transcription factor.

That is, the present invention provides the following:
[1] A modified microorganism containing a genetic modification that suppresses a transcription factor that controls expression of pyruvate dehydrogenase; and
   wherein the microorganism has a production pathway of a C6 compound, and
   wherein the C6 compound is at least one compound selected from the group consisting of adipic acid, hexamethylenediamine, 1,6-hexanediol, 6-aminohexanoic acid, 6-amino-1-hexanol, 6-hydroxyhexanoic acid, 3-oxoadipic acid, 3-hydroxyadipic acid, and 2,3-dehydroadipic acid;
[2] The modified microorganism according to [1], in which the genetic modification that suppresses a transcription factor that controls expression of pyruvate dehydrogenase is one or more selected from:
   a modification that suppresses expression of a gene encoding the transcription factor; and
   a modification that reduces an activity of the transcription factor in comparison to a non-reduced strain;
[3] The modified microorganism according to [1] or [2], in which the modified microorganism belongs to a genus selected from the group consisting of Escherichia, Bacillus, Corynebacterium, Arthrobacter, Brevibacterium, Clostridium, Zymomonas, Pseudomonas, Burkholderia, Streptomyces, Rhodococcus, Synechocystis, Alkalihalobacillus, Saccharomyces, Schizosaccharomyces, Yarrowia, Candida, Pichia, and Aspergillus;
[4] The modified microorganism according to any one of [1] to [3], in which the modified microorganism is Escherichia coli;
[5] The modified microorganism according to any one of [1] to [4], in which the transcription factor that controls expression of pyruvate dehydrogenase is PdhR;
[6] The modified microorganism according to [5], in which
   PdhR is a protein encoded by:
   (A-1) DNA that consists of SEQ ID NO: 128;
   (A-2) DNA that hybridizes under a stringent condition with DNA consisting of a base sequence complementary to a base sequence of SEQ ID NO: 128 and encodes a protein having PdhR activity;
   (A-3) DNA that consists of a base sequence having 80% or more, 85% or more, 88% or more, 90% or more, 93% or more, 95% or more, 97% or more, 98% or more, or 99% or more sequence identity with a base sequence of SEQ ID NO: 128 and encodes a protein having PdhR activity; (A-4) DNA that consists of a base sequence encoding a protein consisting of an amino acid sequence in which 1 to 10, 1 to 7, 1 to 5, or 1 to 3 amino acids are deleted, substituted, inserted, and/or added in an amino acid sequence of a protein encoded by a base sequence of SEQ ID NO: 128, and encodes a protein having PdhR activity; or
   (A-5) DNA that consists of a degenerate isomer of a base sequence of SEQ ID NO: 128, or
   (B-1) a protein that consists of an amino acid sequence of SEQ ID NO: 129;
   (B-2) a protein that consists of an amino acid sequence having 80% or more, 85% or more, 88% or more, 90% or more, 93% or more, 95% or more, 97% or more, 98% or more, or 99% or more sequence identity with an amino acid sequence of SEQ ID NO: 129 and has PdhR activity; or
   (B-3) a protein that consists of an amino acid sequence in which 1 to 10, 1 to 7, 1 to 5, or 1 to 3 amino acids are deleted, substituted, inserted, and/or added in an amino acid sequence of SEQ ID NO: 129 and has PdhR activity; and
[7] A method for producing a C6 compound, the method including a culture step of culturing the modified microorganism according to any one of [1] to [6].

According to the present invention, there are provided a novel modified microorganism having a C6 compound production ability and a novel method for producing a C6 compound.

### BRIEF DESCRIPTION OF THE DRAWINGS

FIG. 1 is a diagram illustrating an example of a biosynthetic pathway of a modified microorganism of the present invention from acetyl-CoA and succinyl-CoA to C6 compounds;

FIG. 2 is a diagram illustrating an amino acid sequence of each enzyme;

FIG. 3 is a diagram illustrating a base sequence of each primer;

FIG. 4 is a diagram illustrating an amino acid sequence of each enzyme;

FIG. 5 is a diagram illustrating an amino acid sequence of each enzyme;

FIG. 6 is a diagram illustrating an amino acid sequence of each enzyme;

FIG. 7 is a diagram illustrating an amino acid sequence of each enzyme;

FIG. 8 is a diagram illustrating a base sequence of each primer;

FIG. 9 is a diagram illustrating a base sequence of each primer;

FIG. 10 is a diagram illustrating a base sequence of each primer;

FIG. 11 is a diagram illustrating a base sequence encoding each enzyme;

FIG. 12 is a diagram illustrating a base sequence of each of a promoter, a linker, and a terminator;

FIG. 13 is a diagram illustrating a base sequence of each primer;

FIG. 14 is a diagram illustrating a base sequence of each primer;

FIG. 15 is a diagram illustrating a base sequence of each primer;

FIG. 16 is a diagram illustrating a base sequence encoding each enzyme;

FIG. 17 is a diagram illustrating a base sequence of each primer; and

FIG. 18 is a diagram illustrating a base sequence encoding an enzyme (PdhR) and an amino acid sequence of the enzyme.

### DETAILED DESCRIPTION

Hereinafter, embodiments of the present invention will be described in detail. The present invention is not limited to the following embodiments, and various conditions can be changed and modified within the scope of the gist of the present invention. In addition, unless otherwise specified, genetic manipulations such as acquisition of DNA and preparation and transformation of a vector described in the present specification can be performed by the methods described in known documents such as Molecular Cloning 4th Edition (Cold Spring Harbor Laboratory Press, 2012), Current Protocols in Molecular Biology (Greene Publishing Associates and Wiley-Interscience), and genetic engineering experimental note (Takaaki Tamura, YODOSHA CO., LTD.). In the present specification, unless otherwise specified, nucleotide sequences are described from the 5′ direction to the 3′ direction. In the present specification, the terms “polypeptide” and “protein” are used interchangeably. The term “modified microorganism” is synonymous with a “genetic recombinant microorganism”, and the term “genetic recombinant microorganism” is also simply referred to as a “recombinant microorganism”.

In the present specification, a numerical value range indicated by “to” represents a range including numerical values described before and after “to” as the minimum value and the maximum value, respectively. In the numerical value range described in stages in the present specification, an upper limit value or a lower limit value of a numerical value range of a certain stage can be arbitrarily combined with an upper limit value or a lower limit value of a range of numerical values of another stage.

A modified microorganism according to the present invention is a microorganism containing a genetic modification that suppresses a transcription factor that controls expression of pyruvate dehydrogenase and having a production pathway of a C6 compound.

In the present specification, the “C6 compound” includes:
adipic acid (CAS No. 124-04-9);
hexamethylenediamine (CAS No. 124-09-4);
1,6-hexanediol (CAS No. 629-11-8);
6-aminohexanoic acid (CAS No. 60-32-2);
6-amino-1-hexanol (CAS No. 4048-33-3);
6-hydroxyhexanoic acid (CAS No. 1191-25-9);
3-oxoadipic acid (CAS No. 689-31-6);
3-hydroxyadipic acid (CAS No. 14292-29-6); and
2,3-dehydroadipic acid (CAS No. 4440-68-0), and

the “C6 compound” refers to one or a plurality of compounds selected from the group consisting of these compounds.

It is understood by those skilled in the art that the “C6 compound” in the present specification can take a neutral or ionized form including any salt form and this form is pH-dependent.

Regarding the term“production pathway of a C6 compound”, the “C6 compound” is at least one compound selected from the group consisting of adipic acid, hexamethylenediamine, 1,6-hexanediol, 6-aminohexanoic acid, 6-amino-1-hexanol, 6-hydroxyhexanoic acid, 3-oxoadipic acid, 3-hydroxyadipic acid, and 2,3-dehydroadipic acid.

In the present specification, as for the C6 compound, the phrase “containing a production pathway” means that a genetic recombinant microorganism according to the present invention can express a sufficient amount of enzyme to allow each reaction stage of the production pathway of the C6 compound to proceed, and can biosynthesize the compound. That is, the recombinant microorganism according to the present invention can express a sufficient amount of enzyme to allow each reaction stage of the production pathway of the C6 compound to proceed, and can biosynthesize the C6 compound. The recombinant microorganism of the present invention may be a microorganism obtained by using a host microorganism intrinsically having an ability to produce a C6 compound, or may be a microorganism being modified so that a host microorganism intrinsically not having an ability to produce the compound has a C6 compound production ability.

In the present specification, the term “endogenous” or “endogenous property” is used to mean that the host microorganism that is not modified by a genetic recombination has a gene or a protein (typically, an enzyme or a transcription factor) encoded by the same, regardless of whether the gene referred to or the protein encoded by the same is functionally expressed to the extent that a predominant biochemical reaction can proceed in the host microorganism.

In the present specification, the term “exogenous” or “exogeneous property” is used to mean that a gene or a nucleic acid sequence is introduced into a host based on the present invention in a case where a pre-genetically recombinant host microorganism does not have a gene to be introduced by the present invention, in a case where a protein (typically, an enzyme or a transcription factor) encoded by the gene is not substantially expressed, or in a case where an amino acid sequence of the protein is encoded by another gene but the endogenous protein is not expressed in an sufficient amount comparable to the amount expressed after the genetic recombination. The terms “exogensou property” and “external property” are used interchangeably in the present specification.

In the present specification, as for a certain compound, the phrase “containing a production pathway” means that a modified microorganism according to the present invention can express a sufficient amount of enzyme to allow each reaction stage of the production pathway of the compound to proceed, and can biosynthesize the compound.

In the present specification, the “host microorganism” means a microorganism that is able to have a production pathway of a target compound. The terms “host microorganism” and “host” are used interchangeably in the present specification. The host microorganism of the present invention is not particularly limited, and may be either a prokaryote or a eukaryote. Any one of a microorganism isolated and preserved in advance, a microorganism newly isolated from nature, and a microorganism subjected to a genetic modification, can be arbitrarily selected.

The host microorganism belongs to, for example, a genus selected from the group consisting of Escherichia, Bacillus, Corynebacterium, Arthrobacter, Brevibacterium, Clostridium, Zymomonas, Pseudomonas, Burkholderia, Streptomyces, Rhodococcus, Synechocystis, Alkalihalobacillus, Saccharomyces, Schizosaccharomyces, Yarrowia, Candida, Pichia, and Aspergillus. Escherichia coli is preferably used as the host microorganism in the present invention.

Accordingly, the modified microorganism according to the present invention belongs to, for example, a genus selected from the group consisting of Escherichia, Bacillus, Corynebacterium, Arthrobacter, Brevibacterium, Clostridium, Zymomonas, Pseudomonas, Burkholderia, Streptomyces, Rhodococcus, Synechocystis, Alkalihalobacillus, Saccharomyces, Schizosaccharomyces, Yarrowia, Candida, Pichia, and Aspergillus, and it is preferably Escherichia coli.

As described above, the modified microorganism according to the present invention contains a genetic modification that suppresses a transcription factor that controls expression of pyruvate dehydrogenase. In the present specification, the term “transcription factor that controls expression of pyruvate dehydrogenase” is also simply referred to as a “transcription factor”.

Examples of the transcription factor that controls expression of pyruvate dehydrogenase include PdhR of Escherichia coli. PdhR is a protein encoded by the pdhR gene of Escherichia coli, and acts as a transcription factor that controls expression of an enzyme gene including a pyruvate dehydrogenase complex. pdhR is also referred to as aceC, genA, and yacB as synonyms. Examples of an amino acid sequence of PdhR include, but are not limited to:
PdhR of Escherichia coli BL21(DE3) strain registered as GenBank Accession No. ACT42013.1; and
PdhR of Escherichia coli K-12 MG1655 strain registered as GenBank Accession No. AAC73224.1. Examples of a base sequence of a pdhR gene-encoding region include, but are not limited to:
pdhR gene of Escherichia coli K-12 MG1655 strain registered as NCBI GeneID 944827.

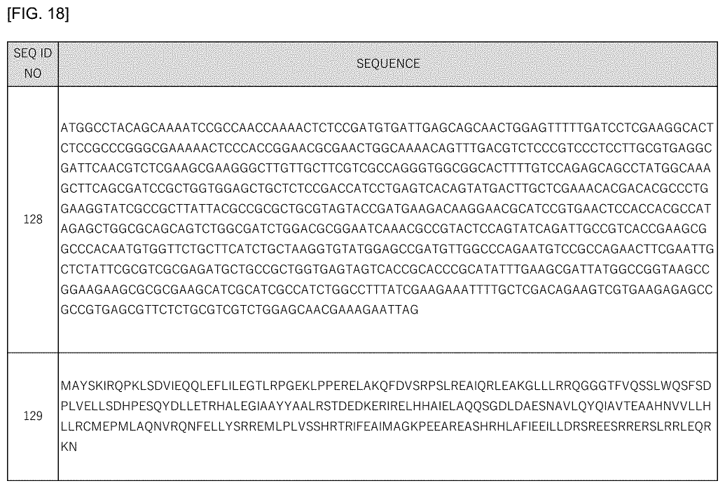

In one aspect, PdhR is a protein encoded by:
(A-1) DNA that consists of SEQ ID NO: 128;
(A-2) DNA that hybridizes under a stringent condition with DNA consisting of a base sequence complementary to a base sequence of SEQ ID NO: 128 and encodes a protein having PdhR activity;
(A-3) DNA that consists of a base sequence having 80% or more, 85% or more, 88% or more, 90% or more, 93% or more, 95% or more, 97% or more, 98% or more, or 99% or more sequence identity with a base sequence of SEQ ID NO: 128 and encodes a protein having PdhR activity;
(A-4) DNA that consists of a base sequence encoding a protein consisting of an amino acid sequence in which 1 to 10, 1 to 7, 1 to 5, or 1 to 3 amino acids are deleted, substituted, inserted, and/or added in an amino acid sequence of a protein encoded by a base sequence of SEQ ID NO: 128, and encodes a protein having PdhR activity; or
(A-5) DNA that consists of a degenerate isomer of a base sequence of SEQ ID NO: 128 (FIG. 18).

In a preferred aspect, PdhR is a protein encoded by:
(A-1) DNA that consists of SEQ ID NO: 128;
(A-2) DNA that hybridizes under a stringent condition with DNA consisting of a base sequence complementary to a base sequence of SEQ ID NO: 128 and encodes a protein having PdhR activity;
(A-3) DNA that consists of a base sequence having 90% or more sequence identity with a base sequence of SEQ ID NO: 128 and encodes a protein having PdhR activity;
(A-4) DNA that consists of a base sequence encoding a protein consisting of an amino acid sequence in which 1 to 10 amino acids are deleted, substituted, inserted, and/or added in an amino acid sequence of a protein encoded by a base sequence of SEQ ID NO: 128, and encodes a protein having PdhR activity; or
(A-5) DNA that consists of a degenerate isomer of a base sequence of SEQ ID NO: 128.

In a more preferred aspect, PdhR is a protein encoded by (A-1) DNA consisting of SEQ ID NO: 128.

In the present specification, the “stringent condition” is, for example, a condition of about “1×SSC, 0.1% SDS, 60° C.”, a more severe condition of about “0.1×SSC, 0.1% SDS, 60° C.”, and a still more severe condition of about “0.1×SSC, 0.1% SDS, 68° C.”.

In another aspect, PdhR is:
(B-1) a protein that consists of an amino acid sequence of SEQ ID NO: 129;
(B-2) a protein that consists of an amino acid sequence having 80% or more, 85% or more, 88% or more, 90% or more, 93% or more, 95% or more, 97% or more, 98% or more, or 99% or more sequence identity with an amino acid sequence of SEQ ID NO: 129 and has PdhR activity; or
(B-3) a protein that consists of an amino acid sequence in which 1 to 10, 1 to 7, 1 to 5, or 1 to 3 amino acids are deleted, substituted, inserted, and/or added in an amino acid sequence of SEQ ID NO: 129 and has PdhR activity (FIG. 18).

In a preferred aspect, PdhR is:
(B-1) a protein that consists of an amino acid sequence of SEQ ID NO: 129;
(B-2) a protein that consists of an amino acid sequence having 90% or more sequence identity with an amino acid sequence of SEQ ID NO: 129 and has PdhR activity; or
(B-3) a protein that consists of an amino acid sequence in which 1 to 10 amino acids are deleted, substituted, inserted, and/or added in an amino acid sequence of SEQ ID NO: 129 and has PdhR activity.

In a more preferred aspect, PdhR is (B-1) a protein consisting of an amino acid sequence of SEQ ID NO: 129.

In the present specification, with regard to the transcription factor, the “genetic modification that suppresses” the transcription factor includes a modification that reduces the activity of the transcription factor in addition to a modification that suppresses the expression of the gene encoding the transcription factor. That is, the modified microorganism of the present invention has been modified so that the expression of the gene encoding the transcription factor is suppressed or that the activity of the transcription factor is reduced. [0038] In the present specification, with regard to the gene encoding the transcription factor, “suppression of expression” also includes “reduction in expression”. In addition, with regard to the transcription factor, “suppression of activity” is synonymous with “suppression of function”, “reduction in function”, and “reduction in activity”, and these terms are used interchangeably. Furthermore, with regard to the microorganism, the terms “non-mutant strain” and “non-reduced strain” are used interchangeably.

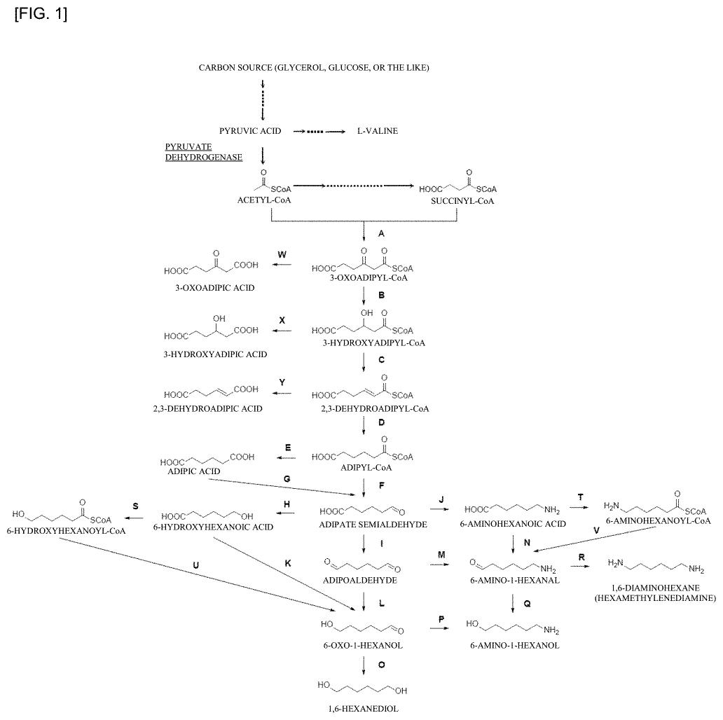

Without intending to be limited to any theory, in the present invention, the transcription factor that controls the expression of pyruvate dehydrogenase negatively controls expression of pyruvate dehydrogenase that catalyzes a reaction from pyruvate to acetyl-CoA. For example, by suppressing the expression of the transcription factor, the suppression of the expression of pyruvate dehydrogenase is removed, and the expression of pyruvate dehydrogenase is promoted. As a result, it is considered that the production of a C6 compound biosynthesized using acetyl-CoA as a precursor is promoted and/or the production of valine biosynthesized using pyruvate as a precursor is reduced (FIG. 1).

In a preferred aspect, the genetic modification that suppresses a transcription factor that controls expression of pyruvate dehydrogenase is one or more selected from:
a modification that suppresses expression of a gene encoding the transcription factor; and
a modification that reduces an activity of the transcription factor in comparison to a non-reduced strain.

In a more preferred aspect, the genetic modification that suppresses a transcription factor that controls expression of pyruvate dehydrogenase is:
a modification that suppresses expression of a gene encoding the transcription factor; or
a modification that reduces an activity of the transcription factor in comparison to a non-reduced strain.

In a still more preferred aspect, the genetic modification that suppresses a transcription factor that controls expression of pyruvate dehydrogenase is a modification that reduces the activity of the transcription factor.

The modification that suppresses the transcription factor can be achieved by, for example, reducing expression of a gene encoding the transcription factor. More specifically, the reduction in the expression of the gene may mean a reduction in a transcription amount of gene (mRNA amount) and/or a reduction in a translation amount of gene (transcription factor amount). The reduction in the expression of the gene also includes a case where a gene is not expressed at all.

The reduction in the expression of the gene may be, for example, a reduction in transcription amount of the gene, a reduction in translation amount of the gene, or a combination thereof. The reduction in the transcription amount can be achieved by, for example, a method of modifying a transcription regulatory region such as a promoter region or an operator region of the gene. The reduction in the transcription amount of the gene can be evaluated by a method known to those skilled in the art, and examples of the method include a quantitative RT-PCR method and a Northern blotting method. The transcription amount of the gene may be reduced to, for example, 90% or less, 80% or less, 70% or less, 60% or less, 50% or less, 40% or less, 30% or less, 20% or less, 10% or less, 5% or less, or 0% in comparison to the non-reduced strain.

Examples of a method of reducing a translation amount include a method of modifying a translation regulatory region such as a ribosome binding site (RBS) and a method of suppressing translation by inserting a riboswitch region upstream of a gene. The riboswitch refers to RNA that selectively binds to a specific low-molecular compound, and the low-molecular compound is referred to as a ligand. A secondary structure is formed by RNA base pairs in the absence of a ligand to affect nucleic acids around the riboswitch. In particular, in a case where a ribosome binding site is included in the downstream of the riboswitch, the access of the ribosome to the ribosome binding site is blocked, which interferes with translation of mRNA of a gene located further downstream. On the other hand, the ribosome can access the ribosome binding site in the presence of a ligand through secondary structure elimination upon ligand binding. Therefore, when a ligand is not added, mRNA of a gene is not translated, and expression of a target gene is suppressed. The reduction in the translation amount of the gene can be evaluated by a method known to those skilled in the art, and examples of the method include a Western blotting method and an ELISA method. The translation amount of the gene may be reduced to, for example, 90% or less, 80% or less, 70% or less, 60% or less, 50% or less, 40% or less, 30% or less, 20% or less, 10% or less, 5% or less, or 0% in comparison to the non-reduced strain.

In addition, the modification that suppresses the transcription factor can be achieved by, for example, a modification that reduces the activity of the transcription factor.

Examples of the modification that reduces the activity of the transcription factor include a modification that reduces or eliminates the activity of the produced transcription factor. The reduction in the activity of the transcription factor can be evaluated by a method known to those skilled in the art, and examples thereof include a gel shift assay. For example, the reduction in the activity of the transcription factor may be 90% or less, 80% or less, 70% or less, 60% or less, 50% or less, 40% or less, 30% or less, 20% or less, 10% or less, 5% or less, or 0% in comparison to the activity of the transcription factor of the non-reduced strain.

The modification that reduces the activity of the transcription factor also includes a modification in which the transcription factor is not produced at all. The production amount of the transcription factor can be evaluated by a method known to those skilled in the art, and examples thereof include a Western blotting method and an ELISA method.

The modification that reduces the activity of the transcription factor is achieved by, for example, disrupting a gene encoding the transcription factor. The disruption of the gene means a modification of the gene so that no active transcription factor is produced, and includes a modification that reduces or eliminates the activity of the produced transcription factor and a modification of the gene so that no transcription factor is produced.

The modification that reduces the activity of the transcription factor can be achieved, for example, by deleting a part or all of a gene-encoding region on chromosomes (that is, disruption of the gene). Furthermore, the entire gene containing sequences before and after a gene on a chromosome may be deleted. As long as the reduction in the activity of the transcription factor can be achieved, the deleted region may be any one of the N-terminus region, the internal region, and the C-terminus region.

In addition, the disruption of the gene can be achieved, for example, by a method of introducing amino acid substitution (missense mutation) or introducing a stop codon (nonsense mutation) into a gene-encoding region on a chromosome, or a method of introducing a frameshift mutation that adds or deletes one or two bases.

Furthermore, the disruption of the gene can be achieved by inserting another sequence into a gene-encoding region on a chromosome. Examples of another sequence include an antibiotic-resistant gene and a transposon, but are not particularly limited as long as the activity is reduced.

The disruption of the gene can be performed by using a method of homologous recombination, and examples of the method include, but are not limited to, a method of using Red reconbinase of λ-phage (Datsenko, Kirill A., and Barry L. Wanner. “One-step inactivation of chromosomal genes in Escherichia coli K-12 using PCR products” Proceedings of the National Academy of Sciences 97.12 (2000): 6640-6645), a method of using a Suicide vector containing a temperature-sensitive replication origin (Blomfield et al., Molecular microbiology 5.6 (1991): 1447-1457), and a method of using a CRISPR-Cas9 system (Jiang, Yu, et al. “Multigene editing in the Escherichia coli genome via the CRISPR-Cas9 system” Appl. Environ. Microbiol. 81.7 (2015): 2506-2514). [0054] In addition, the disruption of the gene can be performed by a mutation treatment. Examples of the mutation treatment include physical treatments such as an X-ray treatment, an ultraviolet ray treatment, and a γ-ray treatment, and chemical treatments with mutagens such as N-methyl-N′-nitro-N-nitrosoguanidine, ethyl methanesulfonate, and methyl methanesulfonate, but are not particularly limited as long as the activity is reduced.

The disruption of the gene can be confirmed by a method known to those skilled in the art, and can be confirmed, for example, by determining the base sequence or the sequence length of the gene-encoding region.

In the present specification, the “non-reduced strain” refers to a strain in which a transcription factor that controls expression of pyruvate dehydrogenase is not suppressed, and is also referred to as a “non-mutant strain”. Examples of the non-reduced strain include, but are not limited to, a wild-type strain or a reference strain of each microbial strain and a derivative strain including a strain obtained by breeding. Examples of the Escherichia coli strain include, but are not limited to, K-12 strain, B strain, C strain, and W strain, and derivative strains thereof such as BL21(DE3) strain, W3110 strain, MG1655 strain, JM109 strain, DH5α strain, and HB101 strain.

In a preferred aspect, the modified microorganism according to the present invention is a modified microorganism containing a C6 compound production pathway in which an enzyme that catalyzes a reaction stage in the C6 compound production pathway is further expressed. An example of the C6 compound production pathway that can be obtained by the modified microorganism of the present invention is illustrated in FIG. 1.

In one aspect, the modified microorganism according to the present invention has, as a gene encoding the “enzyme that catalyzes a reaction stage in a C6 compound production pathway”, at least one selected from the group consisting of:
a gene encoding 3-oxoadipyl-CoA thiolase;
a gene encoding 3-hydroxyadipyl-CoA dehydrogenase;
a gene encoding 3-hydroxyadipyl-CoA dehydratase;
a gene encoding 2,3-dehydroadipyl-CoA reductase;
a gene encoding thioester hydrolase;
a gene encoding CoA-transferase;
a gene encoding phosphate acyltransferase;
a gene encoding phosphotransferase;
a gene encoding acid-thiol ligase;
a gene encoding dehydrogenase;
a gene encoding carboxylic acid reductase;
a gene encoding transaminase (aminotransferase); and
a gene encoding alcohol dehydrogenase.

By having such a gene, the modified microorganism can efficiently produce a C6 compound.

In a preferred aspect, the modified microorganism according to the present invention includes, as a gene encoding the “enzyme that catalyzes a reaction stage in a C6 compound production pathway”, at least:
a gene encoding 3-oxoadipyl-CoA thiolase;
a gene encoding 3-hydroxyadipyl-CoA dehydrogenase;
a gene encoding 3-hydroxyadipyl-CoA dehydratase; and
a gene encoding 2,3-dehydroadipyl-CoA reductase.

By having such a gene, the modified microorganism can efficiently produce adipic acid.

In another preferred aspect, the modified microorganism according to the present invention includes, as a gene encoding the “enzyme that catalyzes a reaction stage in a C6 compound production pathway”, at least:
a gene encoding 3-oxoadipyl-CoA thiolase;
a gene encoding 3-hydroxyadipyl-CoA dehydrogenase;
a gene encoding 3-hydroxyadipyl-CoA dehydratase;
a gene encoding 2,3-dehydroadipyl-CoA reductase;
a gene encoding carboxylic acid reductase; and
a gene encoding alcohol dehydrogenase.

By having such a gene, the modified microorganism can efficiently produce 6-hydroxyhexanoic acid and/or 1,6-hexanediol.

In still another preferred aspect, the modified microorganism according to the present invention includes, as a gene encoding the “enzyme that catalyzes a reaction stage in a C6 compound production pathway”, at least:
a gene encoding 3-oxoadipyl-CoA thiolase;
a gene encoding 3-hydroxyadipyl-CoA dehydrogenase;
a gene encoding 3-hydroxyadipyl-CoA dehydratase;
a gene encoding 2,3-dehydroadipyl-CoA reductase;
a gene encoding carboxylic acid reductase; and
a gene encoding transaminase (aminotransferase).

By having such a gene, the modified microorganism can efficiently produce 6-aminohexanoic acid and/or hexamethylenediamine (1,6-diaminohexane).
3-oxoadipyl-CoA thiolase catalyzes the reaction of Step A in FIG. 1,
3-hydroxyadipyl-CoA dehydrogenase catalyzes the reaction of Step B in FIG. 1,
3-hydroxyadipyl-CoA dehydratase catalyzes the reaction of Step C in FIG. 1,
2,3-dehydroadipyl-CoA reductase catalyzes the reaction of Step D in FIG. 1,
thioester hydrolase catalyzes at least one reaction of Steps E, W, X, and Y in FIG. 1,
CoA-transferase catalyzes at least one reaction of Steps E, W, X, Y, S, and T in FIG. 1,
a combination of phosphate acyltransferase and phosphotransferase catalyzes at least one reaction of Steps E, W, X, Y, S, and T in FIG. 1,
acid-thiol ligase catalyzes at least one reaction of Steps E, W, X, Y, S, and T in FIG. 1,
dehydrogenase catalyzes at least one reaction of Steps F, U, and V in FIG. 1,
carboxylic acid reductase catalyzes at least one reaction of Steps G, I, K, and N in FIG. 1,
transaminase (aminotransferase) catalyzes at least one reaction of Steps J, M, P, and R in FIG. 1, and
alcohol dehydrogenase catalyzes at least one reaction of Steps H, L, O, and Q in FIG. 1.

In a case where the modified microorganism of the present invention expresses a sufficient amount of enzyme that catalyzes a reaction stage in a C6 compound production pathway without introduction of an exogeneous gene, the reaction may proceed by the enzyme encoded by the endogenous gene.

The enzyme that catalyzes each reaction stage in the C6 compound production pathway of the modified microorganism of the present invention may be encoded by a single gene or may be encoded by a plurality of genes.

Hereinafter, the enzyme that catalyzes each reaction included in the C6 compound production pathway will be described with reference to FIG. 1.

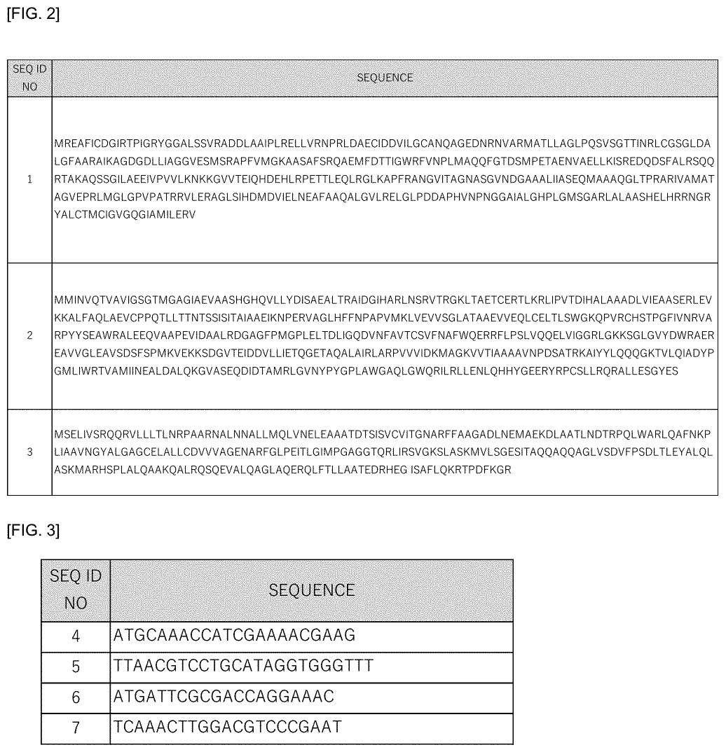

In Step A of FIG. 1, succinyl-CoA and acetyl-CoA are condensed and converted into 3-oxoadipyl-CoA. Examples of the enzyme that can catalyze the present conversion include β-ketothiolase. Furthermore, for example, enzymes classified into the group such as EC 2.3.1.9 (acetoacetyl-CoA thiolase), EC 2.3.1.16 (3-ketoacyl-CoA thiolase), or EC 2.3.1.174 (3-oxoadipyl-CoA thiolase) can be exemplified as enzymes that can have an activity for the present conversion. The enzyme used in the present invention is not limited as long as it has an activity for the present conversion, and examples thereof include 3-oxoadipyl-CoA thiolase. In one aspect, Escherichia coli-derived PaaJ consisting of an amino acid sequence set forth in SEQ ID NO: 1 is used (FIG. 2).

In Step B of FIG. 1, 3-oxoadipyl-CoA is converted into 3-hydroxyadipyl-CoA. Examples of the enzyme that can catalyze the present conversion include oxidoreductase classified into the group of EC 1.1.1. For example, enzymes classified into the group of EC 1.1.1.35 (3-hydroxyacyl-CoA dehydrogenase), EC 1.1.1.36 (acetoacetyl-CoA dehydrogenase), EC 1.1.1.157 (3-hydroxybutanoyl-CoA dehydrogenase), EC 1.1.1.211 (long chain 3-hydroxyacyl-CoA dehydrogenase), or EC 1.1.1.259 (3-hydroxypimeloyl-CoA dehydrogenase) can be exemplified as enzymes that can have an activity for the present conversion. The enzyme used in the present invention is not limited as long as it has an activity for the present conversion, and examples thereof include 3-hydroxyadipyl-CoA dehydrogenase. In one aspect, Escherichia coli-derived PaaH consisting of an amino acid sequence set forth in SEQ ID NO: 2 is used.

In Step C of FIG. 1, 3-hydroxyadipyl-CoA is converted into 2,3-dehydroadipyl-CoA. Examples of the enzyme that can catalyze the present conversion include hydrolyase classified into the group of EC 4.2.1. For example, enzymes classified into the group of EC 4.2.1.17 (enoyl-CoA hydratase), EC 4.2.1.55 (3-hydroxybutanoyl-CoA dehydratase), or EC 4.2.1.74 (long chain enoyl-CoA hydratase) can be exemplified as enzymes that can have an activity for the present conversion. The enzyme used in the present invention is not limited as long as it has an activity for the present conversion, and examples thereof include 3-hydroxyadipyl-CoA dehydratase. In one aspect, Escherichia coli-derived PaaF consisting of an amino acid sequence set forth in SEQ ID NO: 3 is used (FIG. 2). In addition, as another aspect, as the enzyme, PaaF(L3) of 3-hydroxyadipyl-CoA dehydratase encoded by a base sequence (783 bp) of a PCR amplification product using a nucleotide set forth in SEQ ID NO: 4 and a nucleotide set forth in SEQ ID NO: 5 as primers (FIG. 3) and using chromosomal DNA of Burkholderia sp. LEBP-3 strain as a template, is used. Burkholderia sp. LEBP-3 strain (hereinafter, also abbreviated as “L3 strain”) was internationally deposited at National Institute of Technology and Evaluation, Patent Microorganisms Depositary (NPMD) on Dec. 4, 2020, under “Accession No: NITE BP-03334” (Receipt No: NITE ABP-03334).

Here, as the DNA polymerase used for preparing the PCR amplification product, a known DNA polymerase in the art is used, and examples thereof include, but are not limited to, Taq DNA polymerase, hot start adjusted DNA polymerase, and proofreading DNA polymerase having 3′-5′ exonuclease activity in addition to polymerase activity. In a preferred aspect of the conditions for preparing the PCR amplification product, a PCR amplification product is prepared by performing 30 cycles of a treatment with an amount of 25 μL of a solution under conditions of a heat treatment at 98° C. for 10 seconds, annealing at 55° C. for 15 seconds, and elongation at 72° C. and 5 sec/kb using a 1 μM of specific nucleotide as each of a forward primer and a reverse primer, and using PrimeSTAR Max DNA Polymerase (trade name, manufactured by TAKARA BIO INC.) as an enzyme.

In Step D of FIG. 1, 2,3-dehydroadipyl-CoA is converted into adipyl-CoA. Examples of the enzyme that can catalyze the present conversion include oxidoreductase classified into the group of EC 1.3.1. For example, enzymes classified into the group of EC 1.3.1.8 (acyl-CoA dehydrogenase (NADP⁺)), EC 1.3.1.9 (enoyl-ACP reductase (NADH)), EC 1.3.1.38 (trans-2-enoyl-CoA reductase (NADP⁺)) (Ter), EC 1.3.1.44 (trans-2-enoyl-CoA reductase (NAD⁺)), EC 1.3.1.86 (crotonyl-CoA reductase), EC 1.3.1.93 (long chain acyl-CoA reductase), or EC 1.3.1.104 (enoyl-ACP reductase (NADPH)) can be exemplified as enzymes that can have an activity for the present conversion. The enzyme used in the present invention is not limited as long as it has an activity for the present conversion, and examples thereof include 2,3-dehydroadipyl-CoA reductase.

The 2,3-dehydroadipyl-CoA reductase used in the present invention is not particularly limited, and as a typical enzyme, for example, Acinetobacter baylyi-derived enzyme DcaA (Kallscheuer, Nicolai, et al. “Improved production of adipate with Escherichia coli by reversal of β-oxidation” Applied microbiology and biotechnology 101.6 (2017): 2371-2382.), and enoyl-CoA reductase derived from a biological species such as Candida tropicalis, Euglena gracilis, Clostridium beijerinckii, and Yarrowia lipolytica (JP 2011-512868 A) have been reported to have 2,3-dehydroadipyl-CoA reductase activity, and can also be used in the present invention. In addition as still another aspect, as the enzyme, MmgC(L3) of 2,3-dehydroadipyl-CoA reductase encoded by a base sequence (1155 bp) of a PCR amplification product using a nucleotide set forth in SEQ ID NO: 6 and a nucleotide set forth in SEQ ID NO: 7 as primers (FIG. 3) and using chromosomal DNA of Burkholderia sp. L3 strain as a template, can be used.

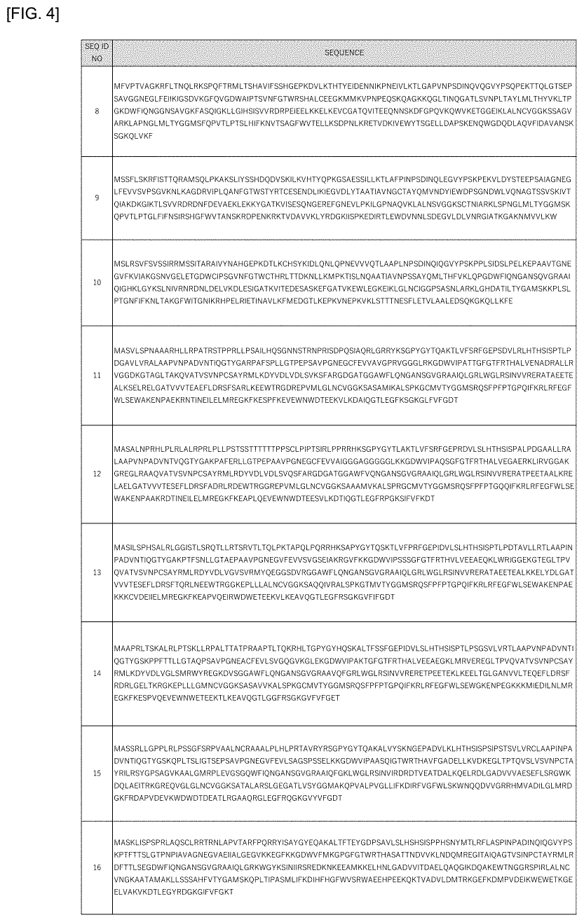

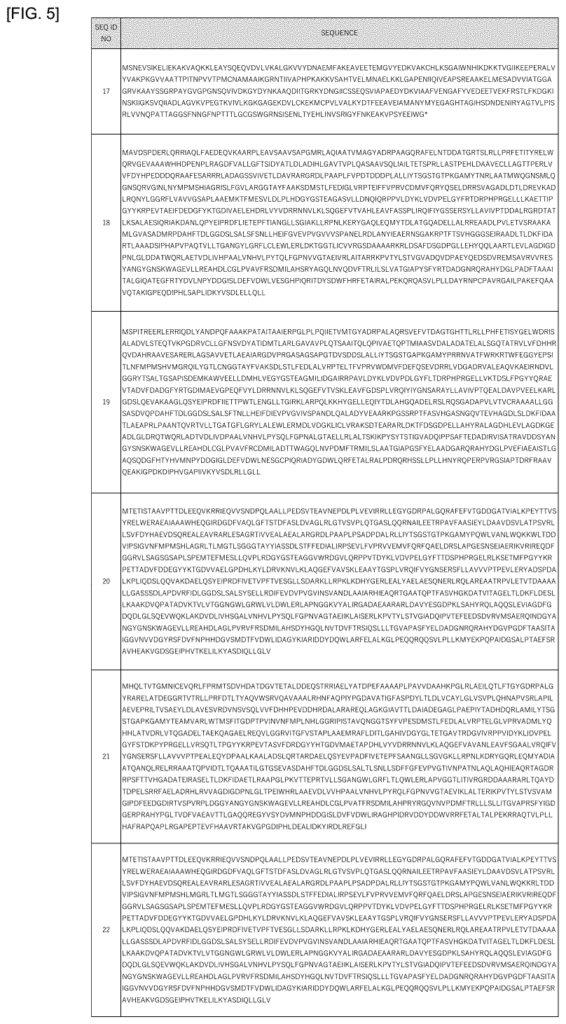

In addition, for example, an enzyme derived from a biological species such as Candida auris, Kluyveromyces marxianus, Pichia kudriavzevii, Thermothelomyces thermophilus, Thermothielavioides terrestris, Chaetomium thermophilum, Podospora anserina, Purpureocillium lilacinum, or Pyrenophora teres can be used, and at least one of enzymes consisting of amino acid sequences set forth in SEQ ID NOs: 8 to 16 is preferably used (FIG. 4).

In the reactions of Steps E, W, X, and Y in FIG. 1, coenzyme A (CoA) is detached. Examples of the enzyme that can catalyze the present conversion include thioester hydrolase classified into the group of EC 3.1.2. For example, enzymes classified into the group of EC 3.1.2.1 (acetyl-CoA hydrolase) or EC 3.1.2.20 (acyl-CoA hydrolase) can be exemplified as enzymes that can have an activity for the present conversion. The enzyme that can be used in the present invention is not limited as long as it has an activity for the present conversion.

In addition, examples of another enzyme that can catalyze the reactions of Steps E, W, X, and Y in FIG. 1 include CoA transferase classified into the group of EC 2.8.3. For example, enzymes classified into the group of EC 2.8.3.5 (3-oxoacid CoA transferase), EC 2.8.3.6 (3-oxoadipate CoA-transferase), or EC 2.8.3.18 (succinyl-CoA: acetyl-CoA-transferase) can be exemplified as enzymes that can have an activity for the present conversion. The enzyme that can be used in the present invention is not limited as long as it has an activity for the present conversion.

Furthermore, examples of conversion of another enzyme that can catalyze the reactions of Steps E, W, X, and Y in FIG. 1 include a pathway that transfers the acyl group of acyl-CoA to a phosphate to form an acyl phosphate by phosphate acyltransferases classified into the group of EC 2.3.1 and then undergoes dephosphorylation by phosphotransferases classified into group EC 2.7.2. For example, as acyltransferase, enzymes classified into the group of EC 2.3.1.8 (phosphate acetyltransferase) or EC 2.3.1.19 (butyryl phosphate transferase) can be exemplified as enzymes that can have an activity for the present conversion. As phosphotransferase, enzymes classified into the group of EC 2.7.2.1 (acetate kinase) or EC 2.7.2.7 (butanoate kinase) can be exemplified as enzymes that can have an activity for the present conversion. The enzyme that can be used in the present invention is not limited as long as it has an activity for the present conversion.

In addition, examples of another enzyme that can catalyze the reactions of Steps E, W, X, and Y in FIG. 1 include acid-thiol ligase classified into the group of EC 6.2.1. For example, enzymes classified into the group of EC 6.2.1.1 (acetyl-CoA synthetase), EC 6.2.1.13 (acetyl-CoA synthetase), EC 6.2.1.4 (succinyl-CoA synthetase), EC 6.2.1.5 (succinyl-CoA synthetase), or EC 6.2.1.14 (pimeloyl-CoA synthetase) can be exemplified as enzymes that can have an activity for the present conversion. The enzyme used in the present invention is not limited as long as it has an activity for the present conversion.

In Steps F and M in FIG. 1, adipyl-CoA is converted into adipate semialdehyde. Examples of the enzyme that can catalyze the present conversion include enzymes classified into the group of EC 1.2.1. For example, enzymes classified into the group of EC 1.2.1.10 (acetaldehyde dehydrogenase (acetylation)), EC 1.2.1.17 (glyoxylate dehydrogenase (acylation)), EC 1.2.1.42 (hexadecanal dehydrogenase (acylation)), EC 1.2.1.44 (cinnamoyl-CoA reductase (acylation)), EC 1.2.1.75 (malonyl-CoA reductase (malonic semialdehyde formation)), or EC 1.2.1.76 (succinic semialdehyde dehydrogenase (acylation)) can be exemplified as enzymes that can have an activity for the present conversion because these enzymes catalyze a conversion reaction in which CoA is removed to produce an aldehyde similarly to the present conversion. The enzyme used in the present invention is not limited as long as it has an activity for the present conversion, and for example, sucD derived from Clostridium kluyveri consisting of an amino acid sequence set forth in SEQ ID NO: 17 is used (FIG. 5).

In the reactions of Steps G, I, K, and N in FIG. 1, a carboxyl group is converted into an aldehyde. Examples of the enzyme that can catalyze the present conversion include carboxylic acid reductase (CAR). For example, enzymes classified into the group of EC 1.2.1.30 (carboxylic acid reductase (NADP⁺)), EC 1.2.1.31 (L-aminoadipate semialdehyde dehydrogenase), EC 1.2.1.95 (L-2-aminoadipate reductase), or EC 1.2.99.6 (carboxylic acid reductase) can be exemplified as enzymes that can have an activity for the present conversion because these enzymes catalyze a conversion reaction in which an aldehyde is generated from a carboxylic acid similarly to the present conversion. Typical examples of the biological species from which the enzyme is derived include, but are not limited to, Nocardia iowensis, Nocardia asteroides, Nocardia brasiliensis, Nocardia farcinica, Segniliparus rugosus, Segniliparus rotundus, Tsukamurella paurometabola, Mycobacterium marinum, Mycobacterium neoaurum, Mycobacterium abscessus, Mycobacterium avium, Mycobacterium chelonae, Mycobacterium immunogenum, Mycobacterium smegmatis, Serpula lacrymans, Heterobasidion annosum, Coprinopsis cinerea, Aspergillus flavus, Aspergillus terreus, Neurospora crassa, and Saccharomyces cerevisiae. The enzyme used in the present invention is not limited as long as it has an activity for the present conversion, and for example, at least one of enzymes consisting of an amino acid sequence set forth in any of SEQ ID NOs: 18 to 22 (FIG. 5) may be used, at least one of enzyme MaCar derived from Mycobacterium abscessus consisting of an amino acid sequence set forth in SEQ ID NO: 20 and MaCar(m) that is a variant of MaCar consisting of an amino acid sequence set forth in SEQ ID NO: 22 is preferably used, and at least MaCar(m) consisting of an amino acid sequence set forth in SEQ ID NO: 22 is more preferably used.

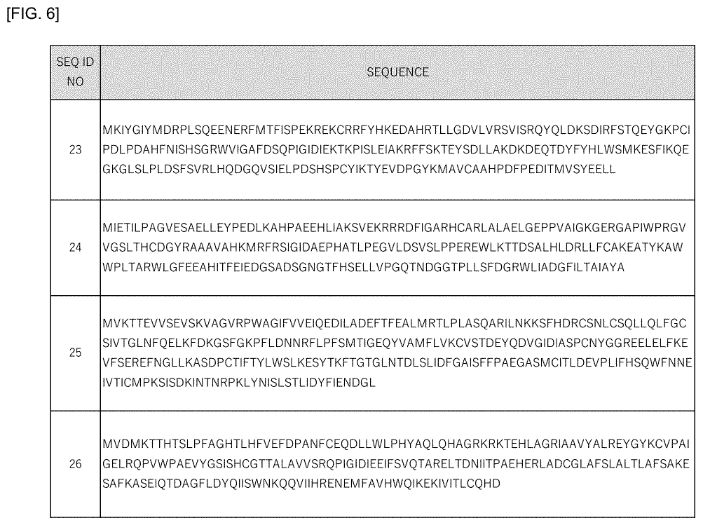

In addition, the carboxylic acid reductase can be converted into an active holoenzyme by being phosphopantetinylated (Venkitasubramanian et al., Journal of Biological Chemistry, Vol. 282, No. 1, 478-485 (2007)). The phosphopantetinylation is catalyzed by a phosphopantetheinyl transferase (PT). Examples of the enzyme that can catalyze the present reaction include enzymes classified into EC 2.7.8.7. Therefore, the microorganism of the present invention may be further modified to increase an activity of the phosphopantetheinyl transferase. Examples of a method of increasing the activity of the phosphopantetheinyl transferase include, but are not limited to, a method of introducing an exogenous phosphopantetheinyl transferase and a method of enhancing expression of an endogenous phosphopantetheinyl transferase. The enzyme used in the present invention is not particularly limited as long as it has a pheosphopantetheinyl group transfer activity, and examples thereof include EntD of Escherichia coli, Sfp of Bacillus subtilis, Npt of Nocardia iowensis (Venkitasubramanian et al., Journal of Biological Chemistry, Vol. 282, No.1, 478-485 (2007)), and Lys5 of Saccharomyces cerevisiae (Ehmann et al., Biochemistry 38.19 (1999): 6171-6177). The enzyme used in the present invention is not limited as long as it has an activity for the present conversion, and for example, at least one of enzymes consisting of amino acid sequences set forth in SEQ ID NOs: 23 to 26 may be used (FIG. 6), and Npt of Nocardia iowensis derived from Nocardia iowensis consisting of an amino acid sequence set forth in SEQ ID NO: 24 is preferably used.

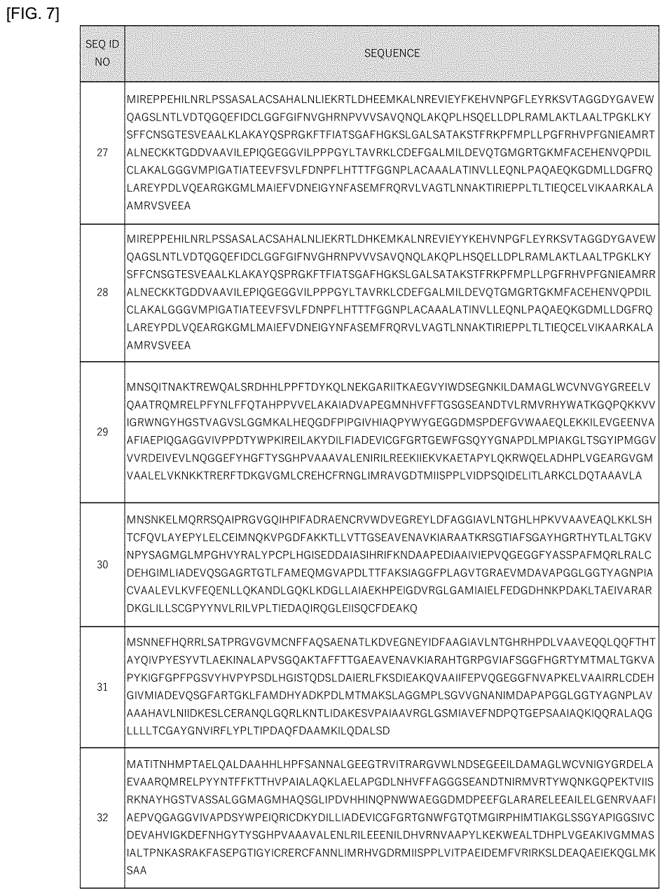

The reactions of Steps J, M, P, and R in FIG. 1 are transamination reactions. Examples of the enzyme that can catalyze the conversion include transaminase (aminotransferase) classified in the group of EC 2.6.1. For example, enzymes classified into the group of EC 2.6.1.19 (4-aminobutanoate-2-oxoglutarate transaminase), EC 2.6.1.29 (diamine transaminase), or EC 2.6.1.48 (5-aminovalerate transaminase) can be exemplified as enzymes that can have an activity for the present conversion. The enzyme used in the present invention is not particularly limited as long as it has the conversion activity of each step, for example, YgjG that is putrescine aminotransferase of Escherichia coli reported to transaminate cadaverine and spermidine (Samsonova., et al., BMC microbiology 3.1 (2003): 2), SpuC that is putrescine aminotransferase of the genus Pseudomonas (Lu et al., Journal of bacteriology 184.14 (2002): 3765-3773, Galman et al., Green Chemistry 19.2 (2017): 361-366), GABA aminotransferase GabT of Escherichia coli, and PuuE may also be used. Furthermore, it is reported that an ω-transaminase derived from a biological species such as Ruegeria pomeroyi, Chromobacterium violaceum, Arthrobacter citreus, Sphaerobacter thermophilus, Aspergillus fischeri, Vibrio fluvialis, Agrobacterium tumefaciens, or Mesorhizobium loti also has a transamination activity to a diamine compound such as 1,8-diaminooctane and 1,10-diaminodecane, and these enzymes may be used in the present invention (Sung et al., Green Chemistry 20.20 (2018): 4591-4595, Sattler et al., Angewandte Chemie 124.36 (2012): 9290-9293). The enzyme used in the present invention is not limited as long as it has an activity for the present conversion, and for example, at least one of enzymes consisting of amino acid sequences set forth in SEQ ID NOs: 27 to 32 may be used (FIG. 7). Examples of a typical amino group donor include, but are not limited to, L-glutamic acid, L-alanine, and glycine.

In Steps H, L, O, and Q in FIG. 1, aldehydes are converted into alcohols. Examples of the enzyme that can catalyze the present conversion include oxidoreductase classified into the group of EC 1.1.1. For example, enzymes classified in the group of EC 1.1.1.1 (alcohol dehydrogenase), EC 1.1.1.2 (alcohol dehydrogenase (NADP⁺)), or EC 1.1.1.71 (alcohol dehydrogenase [NAD(P)⁺]) can be exemplified as enzymes that can have an activity for the present conversion because these enzymes catalyze a reaction in which an aldehyde is converted into an alcohol similarly to the present conversion. The enzyme used in the present invention is not limited as long as it has an activity for the present conversion, and for example, is Ahr derived from Escherichia coli set forth in SEQ ID NO: 32 (FIG. 7).

In the reactions of Steps S and T in FIG. 1, CoA is added to a carboxyl group. Examples of the enzyme that can catalyze the present conversion include CoA transferase classified into the group of EC 2.8.3; a combination of phosphate acyltransferase classified into the group of EC 2.3.1 and phosphotransferase classified into the group of EC 2.7.2; and acid-thiol ligase classified into the group of EC 6.2.1. The enzyme used in the present invention is not limited as long as it has an activity for the present conversion.

The gene encoding the enzyme that can be used in the present invention may be derived from an organism other than the exemplified organism or artificially synthesized, and may be any gene that can express a substantial enzyme activity in a microorganism.

In addition, the amino acid sequence of the enzyme or the base sequence of the gene encoding the enzyme that can be used in the present invention can contain all mutations that can occur in nature and/or artificially introduced mutations and modifications as long as a substantial enzyme activity can be expressed by the microorganism, and can contain, for example, mutations such as deletion, substitution, insertion, and addition. For example, the enzyme may have an amino acid sequence in which 1 or more, preferably 1 to 20, more preferably 1 to 10, still more preferably 1 to 7, still more preferably 1 to 5, and particularly preferably 1 to 3 amino acids are deleted, substituted, inserted, and/or added to the amino acid sequence of the enzyme described above.

In addition, it is known that there are extra codons in various codons encoding a specific amino acid, and therefore, alternative codons that are to be finally translated to the same amino acid may also be used in the present invention. That is, since a genetic code degenerates, a plurality of codons can be used to encode a specific amino acid, such that the amino acid sequence can be encoded by an arbitrary set of similar DNA oligonucleotides. Since most organisms are known to preferentially use a subset of a specific codon (optimal codon) (Gene, Vol. 105, pp. 61-72, 1991, and the like), performing of “codon optimization” can also be useful in the present invention.

Therefore, the microorganism according to the present invention can have a base sequence having, for example, 80% or more, 85% or more, 88% or more, 90% or more, 93% or more, 95% or more, 97% or more, 98% or more, or 99% or more sequence identity with the base sequence of the enzyme gene under a condition in which a substantial enzyme activity can be expressed. Alternatively, the microorganism can have a gene encoding an amino acid sequence having, for example, 80% or more, 85% or more, 88% or more, 90% or more, 93% or more, 95% or more, 97% or more, 98% or more, or 99% or more sequence identity with the amino acid sequence of the enzyme.

In the present specification, a proportion (%) of a “sequence identity” of a comparative amino acid sequence to a reference amino acid sequence is defined as a percentage of amino acid residues in the comparative sequence that are identical to the amino acid residues in the reference sequence when the sequences are aligned so that the identity between the two sequences is maximized, and if necessary, a gap is introduced into one or both of the two sequences. In this case, a conservative substitution is not considered as a part of the sequence identity. The sequence identity can be determined by using publicly available computer software, for example, using an alignment search tool such as Basic Local Alignment Search Tool (BLAST). In an alignment, those skilled in the art can determine appropriate parameters to obtain the maximum alignment of the comparative sequence. The “sequence identity” of the nucleotide sequence can also be determined by a similar method.

In the present invention, introduction of the enzyme gene involved in the C6 compound biosynthesis into a host microorganism as an “expression cassette” results in a more stable and high level of enzyme activity. In the present specification, the “expression cassette” refers to a nucleotide having a nucleic acid sequence that is functionally linked to a nucleic acid to be expressed or a gene to be expressed and regulates transcription and translation. Typically, the expression cassette of the present invention has a promoter sequence on the 5′ upstream from the encoding sequence, a terminator sequence on the 3′ upstream, and an optionally additional normal regulatory element in a functionally linked state, and in such a case, a nucleic acid to be expressed or a gene to be expressed is introduced into a microorganism.

The promoter is defined as a DNA sequence that allows RNA polymerase to bind to DNA to initiate RNA synthesis, regardless of whether the promoter is a constitutive expression promoter or an inductive expression promoter. A strong promoter is a promoter that initiates mRNA synthesis at a high frequency, and is also preferably used in the present invention. In Escherichia coli, a major operator and promoter region of a lac system, a trp system, a tac or trc system, or λ-phage, a control region for fd coat protein, a promoter for a glycolytic enzyme (for example, 3-phosphoglycerate kinase and glyceraldehyde-3-phosphate dehydrogenase), glutamate decarboxylase A, or serine hydroxymethyltransferase, a promoter region of RNA polymerase derived from T7 phage, and the like can be used. As the terminator, a T7 terminator, an rrnBT1T2 terminator, a lac terminator, and the like can be used. In addition to the promoter and terminator sequences, examples of other regulatory elements include a selection marker, an amplification signal, and a replication point. A preferred regulatory sequence is described in, for example, “Gene Expression Technology: Methods in Enzymology 185” Academic Press (1990)”.

The expression cassette described above is incorporated into a vector comprised of, for example, a plasmid, a phage, a transposon, an IS element, a fosmid, a cosmid, or a linear or cyclic DNA and is inserted into a host microorganism. A plasmid and a phage are preferred. The vector may be self-replicated in a host microorganism or may be replicated by a chromosome. Examples of a preferred plasmid include pLG338, pACYC184, pBR322, pUC18, pUC19, pKC30, pRep4, pHS1, pKK223-3, pDHE19.2, pHS2, pPLc236, pMBL24, pLG200, pUR290, pIN-III113-B1, λgt11, and pBdCI of Escherichia coli. Other usable plasmids and the like are described in “Gene Cloning and DNA analysis 7th edition”, Wiley-Blackwell (2016). The expression cassette can be introduced into the vector by a conventional method including cutting with an appropriate restriction enzyme, cloning, and ligation. Each expression cassette may be located on one vector or on two or more vectors.

After the vector of the present invention, containing the expression cassette, is constructed as described above, a conventional method can be used as a method that can be applied for introduction of the vector into a host microorganism. Examples of the method include, but are not limited to, a calcium chloride method, an electroporation method, and a conjugation transfer method, and a suitable method can be selected.

The modified microorganism of the present invention may contain any genetic modification as long as the microorganism contains a genetic modification that suppresses a transcription factor that controls expression of pyruvate dehydrogenase and has a C6 compound production pathway. For example, such modification includes, but are not limited to, enhancement of gene expression, suppression of gene expression, enhancement of enzyme activity, suppression of enzyme activity, enhancement of transcription factor activity, and suppression of transcription factor activity.

The modified microorganism obtained as described above is cultured and maintained under conditions suitable for its growth and/or maintenance in order to produce a target C6 compound. A medium composition, culture conditions, and culture duration suitable for transformants derived from various host microorganisms can be easily selected or optimized by those skilled in the art.

Therefore, a second aspect of the present invention relates to a method for producing a target compound, the method including a culture step of culturing the modified microorganism described above. Specifically, the production method includes a culture step of culturing the modified microorganism described above to obtain a culture of the modified microorganism and/or an extract of the culture.

In the culture step, a culture containing microbial cells is obtained by culturing the modified microorganism in a medium containing a carbon source and a nitrogen source. The modified microorganism according to the present invention is cultured under conditions suitable for the compound production as well as the growth and maintenance of the microorganism, and suitable culture medium composition, culture duration, and culture conditions can be easily set by those skilled in the art.

Examples of the carbon source include D-glucose, sucrose, lactose, fructose, maltose, oligosaccharides, polysaccharides, starch, cellulose, rice bran, blackstrap molasses, fats and oils (for example, soybean oil, sunflower oil, peanut oil, palm oil, and the like), fatty acids (for example, palmitic acid, linoleic acid, oleic acid, linolenic acid, and the like), alcohols (for example, glycerol, ethanol, and the like), organic acids (for example, acetic acid, lactic acid, succinic acid, and the like), corn decomposition liquids, and cellulose decomposition liquids. Preferably, the carbon source is D-glucose, sucrose, or glycerol. These carbon sources can be used solely or as a mixture.

The compound produced using a raw material derived from biomass can be clearly distinguished from a synthetic raw material derived from, for example, petroleum, natural gas, or coal, by measurement of a biobased carbon content based on Carbon-14 (radiocarbon) analysis defined in ISO 16620-2 or ASTM D6866.

Examples of the nitrogen source include nitrogen-containing organic compounds (for example, peptone, casamino acid, tryptone, a yeast extract, a meat extract, a malt extract, a corn steep liquor, soy flour, an amino acid, urea, and the like) and inorganic compounds (for example, an aqueous ammonia solution, ammonium sulfate, ammonium chloride, ammonium phosphate, ammonium carbonate, sodium nitrate, ammonium nitrate, and the like). These nitrogen sources can be used solely or as a mixture.

In addition, the medium may contain a corresponding antibiotic in a case where a modified microorganism expresses a useful additional trait, for example, in a case where a modified microorganism contains a marker resistant to an antibiotic. Therefore, a risk of contamination by various bacteria during culture is reduced. Examples of the antibiotic include, but are not limited to, a β-lactam antibiotic such as ampicillin, an aminoglycoside antibiotic such as kanamycin, a macrolide antibiotic such as erythromycin, a tetracycline antibiotic, and chloramphenicol.

In a case where the modified microorganism cannot assimilate the carbon sources such as cellulose and polysaccharides, the modified microorganism can be adapted to produce a target compound using these carbon sources by a known genetic engineering technique such as introduction of an exogeneous gene. Examples of the exogeneous gene include a cellulase gene and an amylase gene.

The culture may be batch or continuous. In addition, in any case, an additional carbon source or the like may be supplied at an appropriate time point of culture. Furthermore, the culture may be performed while controlling conditions such as a preferred temperature, oxygen concentration, and pH. For example, a suitable culture temperature of a general transformant derived from Escherichia coli is usually 15° C. to 55° C., preferably 25° C. to 40° C. In a case where the host microorganism is aerobic, shaking (flask culture or the like), agitation/aeration (jar fermenter culture or the like) may be performed to ensure a suitable oxygen concentration during fermentation. These culture conditions can be easily set by those skilled in the art.

The production method according to the present invention preferably further includes a mixing step of mixing the culture and/or the extract of the culture with a substrate compound to obtain a mixed solution.

In the culture and/or the mixed solution, a target compound is produced as a result of the reaction. Therefore, in a more preferred aspect, the production method according to the present invention further includes a recovery step of recovering the target compound from the culture and/or the mixed solution.

The step of recovering the target compound from the culture is performed by any method intended for separation and/or purification. Examples thereof include, but are not limited to, centrifugation, membrane filtration, membrane separation, crystallization, extraction, distillation, adsorption, phase separation, ion exchange, and various chromatographic methods. The separation and/or purification may be performed by selecting one type of technique or by combining a plurality of techniques.

As described above, according to the present invention, it is possible to provide a novel modified microorganism having a C6 compound production ability and a novel method for producing a C6 compound. In addition, the genetic modification that suppresses a transcription factor that controls expression of pyruvate dehydrogenase is contained in the host microorganism, such that production of valine that is a by-product can be suppressed, and/or production of adipic acid in the production pathway of the C6 compound can be promoted, thereby obtaining a modified microorganism having an excellent C6 compound production ability. C6 compounds can be efficiently produced as the target compound by the modified microorganism or the production method according to the present invention. In addition, various target compounds can be obtained by performing conversion using an additional enzyme according to a desired target compound. Furthermore, since the modified microorganism and/or the production method according to the present invention has an excellent production ability of a target compound, it is expected that the target compound can be produced on an industrial scale. The C6 compound produced by the modified microorganism and/or the production method according to the present invention can used, in particular, as a monomer raw material for producing a polymer.

Although the embodiments for implementing the present invention have been exemplified above, the embodiments described above are merely examples, and are not intended to limit the scope of the invention. The embodiments described above can be implemented in various other forms, and various omissions, substitutions, and modifications can be made without departing from the gist of the invention.

### EXAMPLES

Hereinafter, the present invention will be described based on examples, and the present invention is not limited these examples.

LEBP-3 strain of the genus Burkholderia (hereinafter, also abbreviated as “L3 strain”) is internationally deposited at National Institute of Technology and Evaluation, Patent Microorganisms Depositary (NPMD) on Dec. 4, 2020, under Accession No: NITE BP-03334.

<Construction of Plasmid for Gene Disruption>

A homologous recombination method using pHAK1 (deposited at biotechnology division of National Institute of Technology and Evaluation, Patent Microorganisms Depositary (NPMD) on Mar. 18, 2019, under Receipt No: NITE ABP-02919, International Accession No: NITE BP-02919) was used for disruption of a gene of Escherichia coli, mutation introduction, and gene insertion. pHAK1 includes a temperature-sensitive variant repA gene, a kanamycin resistant gene, and a levansucrase gene sacB derived from Bacillus subtilis. The levansucrase gene lethally acts on a host microorganism under the presence of sucrose. The PCR fragment was amplified using PrimeSTAR Max DNA Polymerase (trade name, manufactured by TAKARA BIO INC.) or KOD FX Neo (trade name, manufactured by Toyobo Co., Ltd.), and the plasmid was prepared using Escherichia coli HST08 strain.

Using the genomic DNA of the Escherichia coli BL21(DE3) strain as a template, a PCR product containing a 5′ homologous region, an encoding region, and a 3′ homologous region of a disruption target gene was obtained. The combinations of the target gene and the primer sequence are shown in Table 1.

TABLE 1
Primer set used in plasmid construction

| Gene name | SEQ ID NO |
|---|---|
| atoB | 33, 34 |
| sucD | 35, 36 |
| ldhA | 37, 38 |
| adhE | 39, 40 |
| yqhD, dkgA | 41, 42 |
| yahK | 43, 44 |
| ahr | 45, 46 |
| pflB | 47, 48 |
| arcA | 49, 50 |
| pdhR | 51, 52 |
| gltA | 53, 54 |

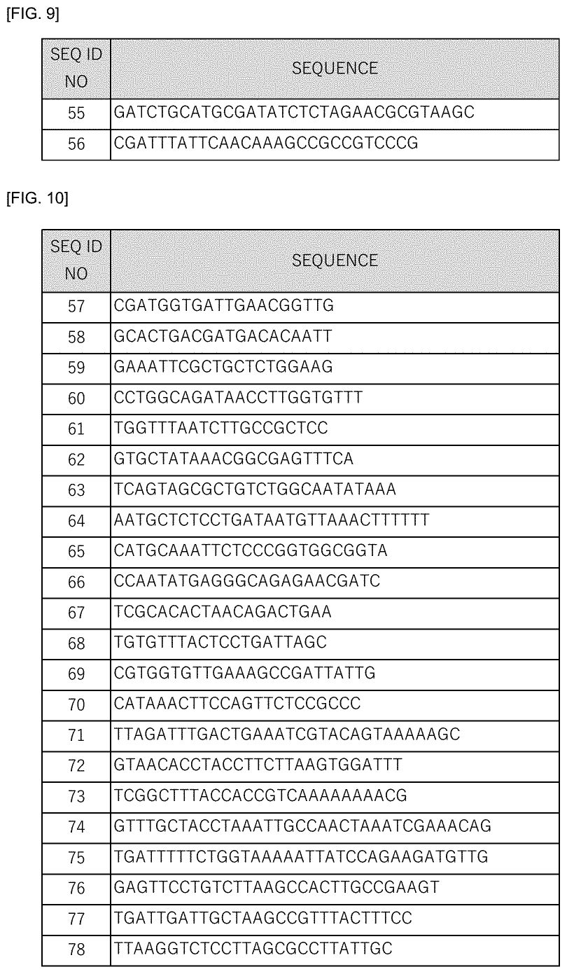

Next, the present PCR product was inserted into the pHAK1 plasmid fragment amplified using primers of SEQ ID NOs: 55 and 56 (FIG. 9) using In-Fusion HD cloning kit (trade name, manufactured by Clontech Laboratories, Inc.) and the PCR product was circularized. Escherichia coli HST08 strain was transformed, and a plasmid was extracted from the obtained transformant.

PCR was performed using the extracted pHAK1 plasmid into which the DNA fragment containing the 5′ homologous region, the encoding region, and the 3′ homologous region of the disruption target gene was inserted as a template and using primers shown in Table 2 and illustrated in FIG. 10, thereby obtaining a pHAK1 plasmid fragment in which the coding region of the disruption target gene was partially or entirely removed and the 5′ homologous region and the 3′ homologous region were contained.

TABLE 2
Primer set used in plasmid construction

| Gene name | SEQ ID NO |
|---|---|
| atoB | 57, 58 |
| sucD | 59, 60 |
| ldhA | 61, 62 |
| adhE | 63, 64 |
| yqhD, dkgA | 65, 66 |
| yahK | 67, 68 |
| ahr | 69, 70 |
| pflB | 71, 72 |
| arcA | 73, 74 |
| pdhR | 75, 76 |
| gltA | 77, 78 |

The obtained plasmid fragment was circularized by terminal phosphorylation and self-ligation. Escherichia coli HST08 strain was transformed, and a plasmid was extracted from the obtained transformant, thereby obtaining a plasmid for gene disruption.

<Construction of Plasmid for Inserting C6 Compound Production Pathway Enzyme Gene>

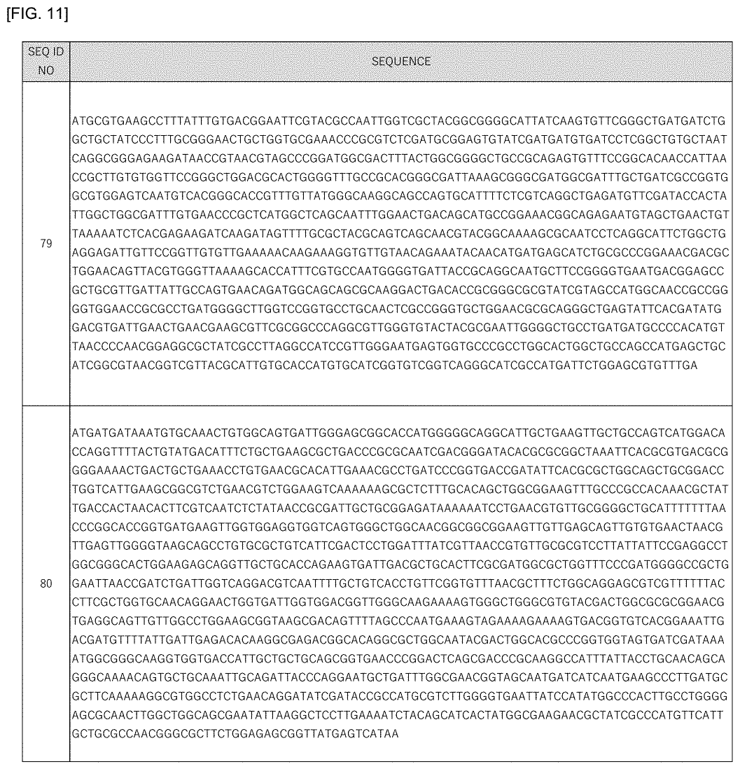

A polynucleotide (SEQ ID NO: 79) encoding PaaJ(Ec) of Eshcherichia coli (SEQ ID NO: 1) was obtained by cloning from Eshcherichia coli W3110 strain (NBRC12713) genomic DNA. A polynucleotide (SEQ ID NO: 80) encoding PaaH(Ec) of Eshcherichia coli (SEQ ID NO: 2) was obtained by cloning from Eshcherichia coli W3110 strain (NBRC12713) genomic DNA (FIGS. 2 and 11).

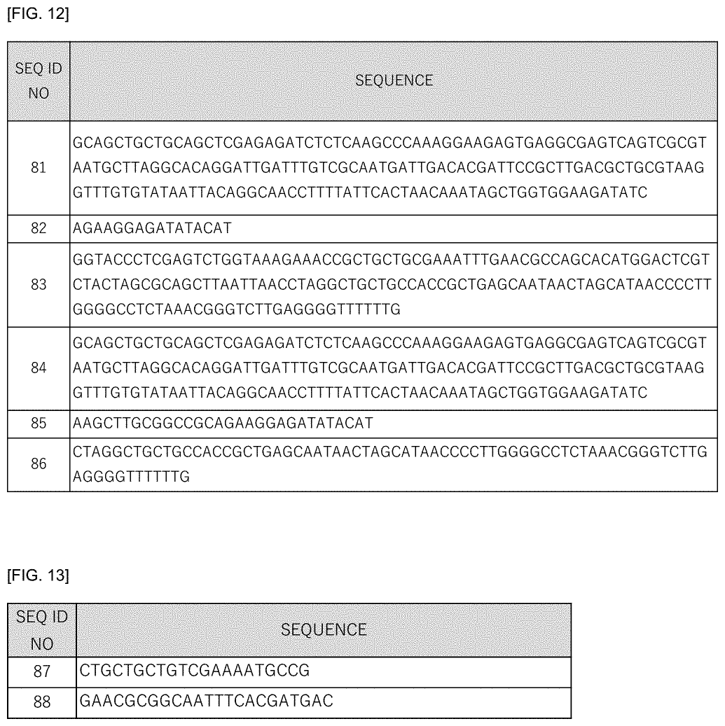

A plasmid for inserting a gene was designed so that the paaJ(Ec) and paaH(Ec) genes were inserted into a pflB gene region as an expression cassette. An expression cassette in which a promoter region (SEQ ID NO: 81), a paaJ(Ec) gene-encoding region, a linker sequence (SEQ ID NO: 82), a paaH-gene encoding region, and a terminator region (SEQ ID NO: 83) were sequentially arranged in this order was incorporated between the 5′ homology region and the 3′ homology region of the pflB gene-encoding region included in the previously constructed plasmid for disrupting the pflB gene (FIG. 12).

The polynucleotide encoding PaaF(L3) of the L3 strain was obtained by optimizing the base sequence for expressing Escherichia coli using the artificial gene synthesis service of Eurofins Genomics. The polynucleotide encoding MmgC(L3) of the L3 strain was obtained by optimizing the base sequence for expressing Escherichia coli using the artificial gene synthesis service of Eurofins Genomics.

A plasmid for inserting a gene was designed so that the optimized sequences of the paaF(L3) and mmgC (L3)-gene encoding regions were inserted into a yahK gene region as an expression cassette. An expression cassette in which a promoter region (SEQ ID NO: 84), a paaF(L3) gene-encoding region, a linker sequence (SEQ ID NO: 85), an mmgC(L3)-gene encoding region, and a terminator region (SEQ ID NO: 86) were sequentially arranged in this order was incorporated between the 5′ homology region and the 3′ homology region of the yahK gene-encoding region included in the previously constructed plasmid for disrupting the yahK gene (FIG. 12).

<Construction of Plasmid for Inserting Mutation-Introduced gltA Gene>

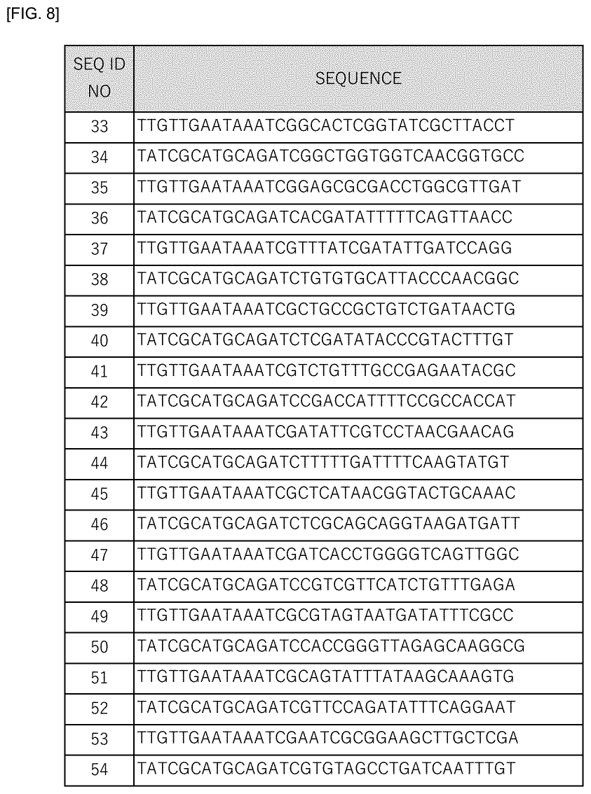

Using the genomic DNA of the Escherichia coli BL21(DE3) strain as a template, a PCR product containing a 5′ homologous region, an encoding region, and a 3′ homologous region of a gltA gene was obtained using primers of SEQ ID NOs: 53 and 54 (FIG. 8).

Next, using In-Fusion HD cloning kit (trade name, manufactured by Clontech Laboratories, Inc.), the present PCR product was inserted into the pHAK1 plasmid fragment amplified using primers of SEQ ID NOs: 55 and 56 to circularize. Escherichia coli HST08 strain was transformed, and a plasmid was extracted from the obtained transformant (FIG. 9).

Using, as a template, the extracted pHAK1 plasmid into which the DNA fragments of the 5′ homology region, the encoding region, and the 3′ homology region of the gltA gene were inserted, PCR was performed using primers set forth in SEQ ID NOs: 87 and 88 (FIG. 13), thereby obtaining a pHAK1 plasmid fragment containing a 5′ homology region, a mutation-introduced gltA gene-encoding region, and a 3′ homology region.

<Construction of Escherichia coli Modified Strain>

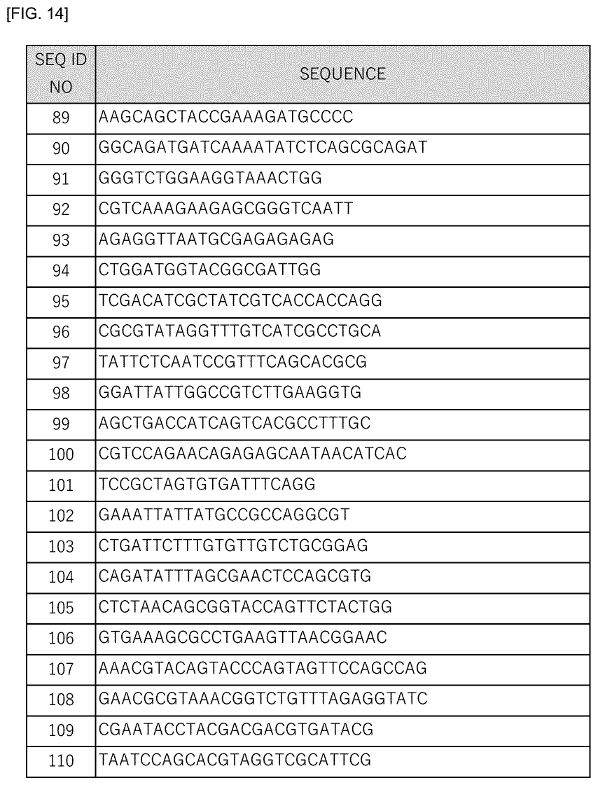

A plasmid for disrupting a desired gene was transformed into the Escherichia coli BL21(DE3) strain by an electroporation method (see Genetic Engineering Laboratory Notebook, by Takaaki Tamura, Yodosha), and then applied to an LB agar medium (10 g/L of tryptone, 5 g/L of yeast extract, 5 g/L of sodium chloride, and 15 g/L of agar powder) containing 100 mg/L of kanamycin sulfate, and incubation was performed at 30° C. overnight to obtain a single colony, thereby obtaining a transformant. The present transformant was inoculated into 1 mL of an LB liquid medium (10 g/L of tryptone, 5 g/L of yeast extract, and 5 g/L of sodium chloride) containing 100 mg/L of kanamycin sulfate with a platinum loop, and shaking culture was performed at 30° C. The obtained culture solution was applied to an LB agar medium containing 100 mg/L of kanamycin sulfate, and incubation was performed at 42° C. overnight. In the obtained colony, a plasmid was inserted into the genome by single crossover. The colony was inoculated into 1 mL of an LB liquid medium with a platinum loop, and shaking culture was performed at 30° C. The obtained culture solution was applied to an LB agar medium containing 20% sucrose, and incubation was performed at 30° C. for 2 days. Disruption or insertion of a desired gene in the obtained colony was confirmed by colony direct PCR using the primer set shown in Table 3 and illustrated in FIG. 14.

The above operation was repeated to construct an Escherichia coli strain containing a plurality of gene disruptions and gene insertions. The constructed Escherichia coli strain is shown in Table 4. In the table, a gene described with A indicates that the enzyme gene is deficient. The “Gene name A::Gene name B” indicates that the gene A region is substituted with a region including the gene B. The “gltA R164L” indicates that the arginine at position 164 of the GltA protein encoded by the gltA gene is substituted with leucine.

TABLE 3
Primer set used for disruption and
insertion target confirmation

| Gene name | SEQ ID NO |
|---|---|
| atoB | 89, 90 |
| sucD | 91, 92 |
| ldhA | 93, 94 |
| adhE | 95, 96 |
| yahD, dkgA | 97, 98 |
| yahK | 99, 100 |
| ahr | 101, 102 |
| pflB | 103, 104 |
| arcA | 105, 106 |
| pdhR | 107, 108 |
| gltA | 109, 110 |

TABLE 4
Modified strains of Escherichia Coli

| Strain No, | Gene disruption and insertion |
|---|---|
| No.048 | ΔatoB, ΔsucD, ΔldhA, ΔadhE, ΔyqhD, ΔdkgA, ΔyahK, Δahr, ΔpflB, ΔarcA |
| | gltA_{R164L} |
| | pflB::paaJ(Ec)-paaH(Ec) |
| | yahK::paaF(L3)-mmgC(L3) |
| No.071 | ΔatoB, ΔsucD, ΔldhA, ΔadhE, ΔyqhD, ΔdkgA, ΔyahK, Δahr, ΔpflB, ΔarcA, ΔpdhR |
| | gltA_{R164L} |
| | pflB::paaJ(Ec)-paaH(Ec) |
| | yahK::paaF(L3)-mmgC(L3) |

<Construction of Plasmid for Expressing C6 Compound Production Pathway Enzyme Gene>

The PCR fragment was amplified using PrimeSTAR Max DNA Polymerase (trade name, manufactured by TAKARA BIO INC.) or KOD FX Neo (trade name, manufactured by Toyobo Co., Ltd.), and the plasmid was prepared using Escherichia coli JM109 strain. For the optimization of the base sequence, GeneArt GeneOptimizer (Software name, Thermo Fisher Scientific Inc.) or an artificial gene synthesis service of Eurofins Genomics. LLC was used.

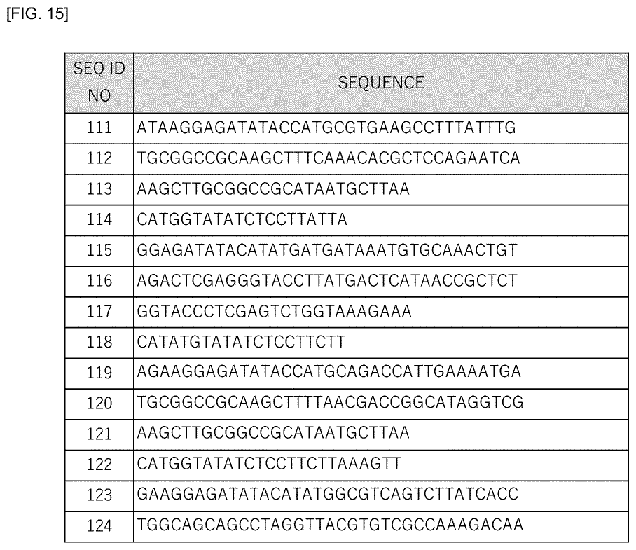

A polynucleotide encoding PaaJ(Ec) of Eshcherichia coli (SEQ ID NO: 1) was obtained by cloning from Eshcherichia coli W3110 strain (NBRC12713) genomic DNA. PCR was performed using oligonucleotides of SEQ ID NOs: 111 and 112 as primers (FIG. 15) to obtain a PCR product containing the encoding region (SEQ ID NO: 79) of the paaJ(Ec) gene (FIG. 11). Next, PCR was performed using pRSFDuet-1 (trade name, manufactured by Merck & Co., Inc.) as a template and oligonucleotides of SEQ ID NOs: 113 and 114 as primers (FIG. 15) to obtain a pRSFDuet-1 fragment. The DNA fragment containing the paaJ(Ec) gene-encoding region and the pRSFDuet-1 fragment were connected using In-Fusion HD cloning kit (trade name, manufactured by Clontech Laboratories, Inc.). Escherichia coli JM109 strain was transformed, and a plasmid was extracted from the obtained transformant. “paaJ(Ec)-pRSFDuet” was obtained as a PaaJ(Ec) expression plasmid.

A polynucleotide encoding PaaH(Ec) of Eshcherichia coli (SEQ ID NO: 2) (FIG. 2) was obtained by cloning from Eshcherichia coli W3110 strain (NBRC12713) genomic DNA. PCR was performed using oligonucleotides of SEQ ID NOs: 115 and 116 as primers (FIG. 15) to obtain a PCR product containing the paaH(Ec) gene-encoding region (SEQ ID NO: 80) (FIG. 11). Next, PCR was performed using the “paaJ(Ec)-pRSFDuet” as a template and oligonucleotides of SEQ ID NOs: 117 and 118 as primers (FIG. 15) to obtain a “paaJ(Ec)-pRSFDuet” fragment. The DNA fragment containing the paaH(Ec) gene-encoding region and the “paaJ(Ec)-pRSFDuet” fragment were connected using In-Fusion HD cloning kit (trade name, manufactured by Clontech Laboratories, Inc.). Escherichia coli JM109 strain was transformed, and a plasmid was extracted from the obtained transformant. “paaJ(Ec)-paaH(Ec)-pRSFDuet” was obtained as a PaaJ(Ec) and PaaH(Ec) co-expression plasmid.

The oligonucleotide set forth in SEQ ID NO: 4 and the oligonucleotide set forth in SEQ ID NO: 5 were used as primers (FIG. 3), and the polynucleotide encoding PaaF(L3) of the L3 strain encoded by the base sequence of the PCR amplification product using the chromosomal DNA of the L3 strain as a template was obtained by optimizing the base sequence for expressing Escherichia coli using the artificial gene synthesis service of Eurofins Genomics. PCR was performed using oligonucleotides of SEQ ID NOs: 119 and 120 as primers (FIG. 15) to obtain a PCR product containing the optimized sequence of the paaF(L3) gene-encoding region. PCR was performed using pETDuet-1 (trade name, manufactured by Merck & Co., Inc.) as a template and oligonucleotides of SEQ ID NOs: 121 and 122 as primers (FIG. 15) to obtain a pETDuet-1 fragment. The DNA fragment containing the paaF(L3)-encoding region and the pETDuet-1 fragment were connected using In-Fusion HD cloning kit (trade name, manufactured by Clontech Laboratories, Inc.). Escherichia coli JM109 strain was transformed, and a plasmid was extracted from the obtained transformant. “paaF(L3)-pETDuet” was obtained as a PaaF(L3) expression plasmid.

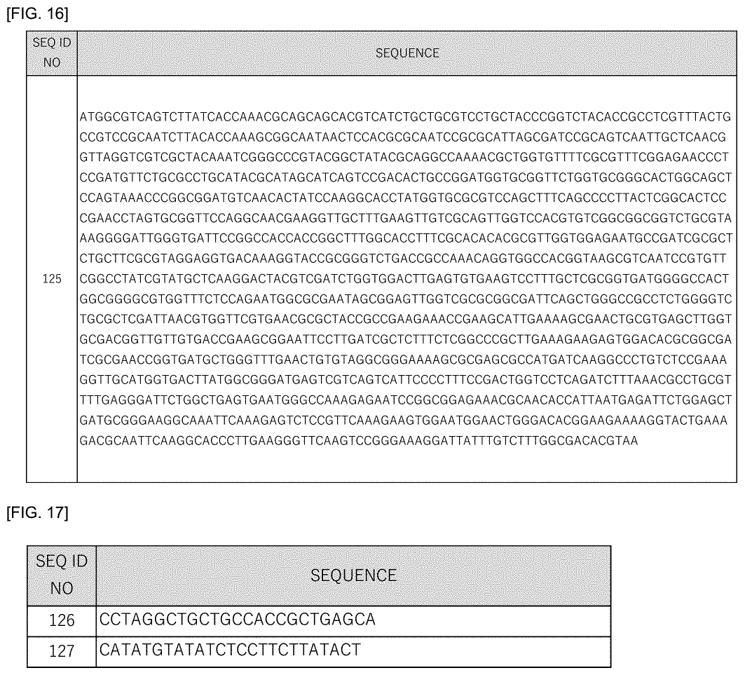

The polynucleotide encoding Ter (SEQ ID NO: 11) of Thermothelomyces thermophilus was obtained by optimizing the base sequence for expressing Escherichia coli using the artificial gene synthesis service of Eurofins Genomics. PCR was performed using oligonucleotides of SEQ ID NOs: 123 and 124 as primers (FIG. 15) to obtain a PCR product containing the ter gene-encoding region (SEQ ID NO: 125) (FIG. 16). PCR was performed using “paaF(L3)-pETDuet” as a template and oligonucleotides of SEQ ID NOs: 126 and 127 as primers (FIG. 17) to obtain a “paaF(L3)-pETDuet” fragment. The DNA fragment containing the ter gene-encoding region and the “paaF(L3)-pETDuet” fragment were connected using In-Fusion HD cloning kit (trade name, manufactured by Clontech Laboratories, Inc.). Escherichia coli JM109 strain was transformed, and a plasmid was extracted from the obtained transformant. “paaF(L3)-ter-pETDuet” was obtained as a PaaF(L3) and Ter co-expression plasmid.

<Adipic Acid Production Test (Comparative Example 1 and Example 1)>

An Escherichia coli modified strain No. 048 or No. 071 was transformed with adipic acid production pathway enzyme expression plasmids “paaJ(Ec)-paaH(Ec)-pRSFDuet” and “paaF(L3)-ter-pETDuet” by an electroporation method, and cultured on an LB agar medium containing 50 mg/L of ampicillin sodium and 30 mg/L of kanamycin sulfate at 37° C. for one day, thereby forming colonies. The colonies were inoculated with one platinum loop into 2 mL of an LB liquid medium (round bottom tube with 14 mL volume) containing 50 mg/L of ampicillin sodium and 30 mg/L of kanamycin sulfate, and shaking culture was performed at 37° C. for 3 to 5 hours, thereby obtaining a pre-culture solution. In a Jar culture device with 250 mL volume (instrument name: Bio Jr. 8, manufactured by ABLE Corporation & Biott Corporation), 0.5 mL of the pre-culture solution was added to a synthetic medium containing 50 mg/L of carbenicillin sodium, 30 mg/L of kanamycin sulfate, and 0.02 mM IPTG (Table 5), and a main culture was performed. Culture conditions are as follows: culture temperature 37° C., culture pH 7.0, pH adjustment 10% (w/v) ammonia water, stirring at 750 rpm, and air flow 1 vvm. When 23 hours had elapsed from the inoculation of the pre-culture solution, 5 mL of a 700 g/L glycerol aqueous solution (35 g of glycerol) was further added, and the bacterial strain SC1 was cultured until 45 hours had elapsed, and the bacterial strain SC2 was cultured until 88 hours had elapsed.

TABLE 5
Synthetic medium composition

| | | |
|---|---|---|
| KH₂PO₄ | 3 | g/L |
| K₂HPO₄ | 6 | g/L |
| (NH₄)₂SO₄ | 2,5 | g/L |
| MgSO₄•7H₂O | 0,3 | g/L |
| CaCl₂•2H₂O | 1 | mg/L |
| FeSO₄•7H₂O | 6 | mg/L |
| MnSO₄•H₂O | 1,5 | mg/L |
| AlCl₃•6H₂O | 1,5 | mg/L |
| CoCl₂ | 0,6 | mg/L |
| ZnSO₄•7H₂O | 0,3 | mg/L |
| Na₂MoO₂•2H₂O | 0,3 | mg/L |
| CuCl₂•2H₂O | 0,2 | mg/L |
| H₃BO₃ | 0,1 | mg/L |
| MOPS (adjusted to pH 7,4 with KOH) | 125 | mM |
| Glycerol | 40 | g/L |

Analysis of the glycerol concentration and the adipic acid concentration in the culture solution was performed under the following conditions using a high performance liquid chromatography Prominence system (manufactured by Shimadzu Corporation).

High Performance Liquid Chromatography (HPLC) Analysis Conditions
Detector: differential refractive index detector
Column: Shim-Pack Fast-OA(G), Fast-OA (two columns connected in series) (manufactured by Shimadzu Corporation)
Oven temperature: 40° C.
Mobile phase: 8 mM methanesulfonic acid aqueous solution
Flow rate: 0.6 mL/min
Injection amount: 10 μL

Analysis of the concentration of valine in the culture medium was performed by GC/MS analysis using trimethylsilyl derivatization. To 40 μL of the culture solution centrifugal supernatant (disodium sebacate was added as an internal standard so as to have a final concentration of 10 mM), 360 μL of an extract solution in which water, methanol, and chloroform were mixed so that water:methanol:chloroform was (5:2:2) (v/v/v), was added, and the mixture was thoroughly stirred with a vortex mixer. After centrifugation (16,000×g, 5 minutes), 40 μL of the supernatant was collected in a separate microtube and centrifugally dried on a centrifugal evaporator for 1 hour. 100 μL of a 20 mg/mL pyridine solution of methoxyamine hydrochloride was added to the obtained dried solid, and the mixture was shaken at 30° C. for 90 minutes. 50 μL of N-methyl-N-trimethylsilyltrifluoroacetamide was added and the mixture was shaken at 37° C. for 30 minutes. The reaction solution was used as a sample for GC/MS measurement, and analysis was performed under the following conditions.

GC/MS Analysis Conditions

Apparatus: GCMS-QP-2020NX (manufactured by Shimadzu Corporation)

Column: fused silica capillary tube inert treatment tube (length: 1 m, outer diameter: 0.35 mm, inner diameter: 0.25 mm, manufactured by GL Sciences Inc.), InertCap 5MS/NP (length: 30 m, inner diameter: 0.25 mm, film thickness: 0.25 μm, manufactured by GL Sciences Inc.)

Sample injection amount: 1 μL

Sample introduction method: Split (split ratio: 25:1)

Vaporization chamber temperature: 230° C.

Carrier gas: helium

Carrier gas linear velocity: 39.0 cm/sec

Oven temperature: kept at 80° C. for 2 minutes→raised at 15° C./min →kept at 325° C. for 13 minutes

Ionization method: electron ionization method (EI)

Ionization energy: 70 eV

Ion source temperature: 230° C.

Scan range: m/z=50 to 500

The results of the metabolite concentration in the culture solution are shown in Table 6. Transformants SC1 and SC2 each contain introduced enzyme genes that each catalyze the reactions corresponding to Steps A, B, C, and D illustrated in FIG. 1, and the reaction corresponding to Step E proceeds by an endogenous enzyme, thereby producing adipic acid. The transformant SC2 using, as a host, the strain No. 071 in which the pdhR gene was disrupted showed a higher accumulation amount of adipic acid and a higher yield thereof with respect to glycerol as compared to the transformant SC1 using, as a host, the strain No. 048 in which the pdhR gene was not disrupted. In addition, accumulation of valine as a by-product was observed in the culture solution of the strain SC1, whereas accumulation of valine was reduced in the culture solution of SC2.

TABLE 6
Adipic acid-producing strain and culture test results

| | | | | Step A | Step B | Step C |
|---|---|---|---|---|---|---|
| | Transformant | Strain | pdhR | (3-oxoadipyl- | (3-hydroxyadipyl- | (3-hydroxyadipyl- |
| | name | No. | gene | CoA thiolase) | CoA dehydrogenase) | CoA dehydratase) |
| Comparative | SC1 | No. 048 | | PaaJ (Ec) | PaaF (Ec) | PaaF (L3) |
| Example 1 | | | | | | |
| Example 1 | SC2 | No. 071 | Δ | PaaJ (Ec) | PaaF (Ec) | PaaF (L3) |

| | | Adipic | | | Adipic |
|---|---|---|---|---|---|
| | Step D | acid | Valine | Consumed | acid |
| | (2,3-dehydroadipyl- | concentration | concentration | glycerol | yield |
| | CoA reductase) | [g/L] | [g/L] | [g/L] | [g/g %] |
| Comparative | mmgC (L3) Ter | 9.2 | 1.98 | 68.9 | 13.4% |
| Example 1 | | | | | |
| Example 1 | mmgC (L3) Ter | 12.4 | 0.03 | 69.7 | 17.7% |

According to the present invention, an efficient production process of, for example, a C6 compound can be provided, and application to production on an industrial scale can be expected.

1. A modified microorganism comprising a genetic modification that suppresses a transcription factor that controls expression of pyruvate dehydrogenase,
wherein the microorganism has a production pathway of a C6 compound, and
wherein the C6 compound is at least one compound selected from the group consisting of adipic acid, hexamethylenediamine, 1,6-hexanediol, 6-aminohexanoic acid, 6-amino-1-hexanol, 6-hydroxyhexanoic acid, 3-oxoadipic acid, 3-hydroxyadipic acid, and 2,3-dehydroadipic acid.

2. The modified microorganism according to claim 1, wherein the genetic modification that suppresses a transcription factor that controls expression of pyruvate dehydrogenase is one or more selected from:
a modification that suppresses expression of a gene encoding the transcription factor. and
a modification that reduces an activity of the transcription factor in comparison to a non-reduced strain.

3. The modified microorganism according to claim 1, wherein the modified microorganism belongs to a genus selected from the group consisting of Escherichia, Bacillus, Corynebacterium, Arthrobacter, Brevibacterium, Clostridium, Zymomonas, Pseudomonas, Burkholderia, Streptomyces, Rhodococcus, Synechocystis, Alkalihalobacillus, Saccharomyces, Schizosaccharomyces, Yarrowia, Candida, Pichia, and Aspergillus.

4. The modified microorganism according to claim 1 wherein the modified microorganism is Escherichia coli.

5. The modified microorganism according to claim 1 wherein the transcription factor that controls expression of pyruvate dehydrogenase is PdhR.

6. A method for producing a C6 compound, the method comprising a culture step of culturing the modified microorganism according to claim 1.
